(19) United States
(12) Patent Application Publication
(10) Pub. No.: US 2026/0060607 A1
(43) Pub. Date: Mar. 5, 2026

(54) METHOD FOR ADAPTIVE IMAGE ACQUISITION IN A SKIN INSPECTION SYSTEM

(71) Applicant: BLUEDROP MEDICAL LIMITED, GALWAY (IE)

(72) Inventors: SIMON KIERSEY, BARNA (IE); CHRISTOPHER MURPHY, DUBLIN (IE); GAVIN CORLEY, ENNIS (IE); KENNETH O'BRIEN, GALWAY (IE); GARRY HIGGINS, GALWAY (IE); ADAM COLLINS, GALWAY (IE)

(21) Appl. No.: 19/320,661

(22) Filed: Sep. 5, 2025

(30) Foreign Application Priority Data
Sep. 5, 2024 (EP) 24198773.4

(51) Int. Cl. A61B 5/00; A61B 5/01; G06T 5/50; G06T 5/92; G06T 7/00; G06V 10/54; G06V 10/56; G06V 10/60; G06V 10/77; G06V 40/10; H04N 23/611; H04N 23/73; H04N 23/74; H04N 23/741

(52) U.S. Cl. CPC A61B 5/447; A61B 5/0037; A61B 5/004; A61B 5/0077; A61B 5/01; A61B 5/445; A61B 5/7275; A61B 5/7435; A61B 5/746; G06T 5/50; G06T 5/92; G06T 7/0012; G06V 10/54; G06V 10/56; G06V 10/60; G06V 10/7715; G06V 40/10; H04N 23/611; H04N 23/73; H04N 23/74; H04N 23/741; A61B 2560/0238; A61B 2576/02; G06T 2200/24; G06T 2207/20208; G06T 2207/30088; G06T 2207/30096

(57) ABSTRACT

A method for operating a skin inspection device is disclosed. The method provides for an adaptive image acquisition process to improve the visibility of detected features. The method comprises capturing a first image of an inspection area and analyzing the first image with a processor to identify a feature of interest. Based on one or more characteristics of the identified feature, the processor determines a set of adjusted image acquisition parameters. A second, feature inspection image is then captured of at least a portion of the inspection area using these adjusted parameters. The adjusted image acquisition parameters are specifically configured to optimize the visibility of the identified feature of interest in the second image, thereby enabling a more detailed and accurate analysis.

### CROSS-REFERENCE TO RELATED APPLICATION

This application claims priority to and the benefit of European Patent Application No. 24198773.4, titled “A skin inspection device for identifying abnormalities” and filed on Sep. 5, 2024, the entire contents of which are hereby incorporated by reference herein.

### FIELD OF THE INVENTION

The present disclosure relates generally to the field of automated imaging and diagnostic systems. More specifically, the invention pertains to a method for operating a skin inspection system that utilizes a feedback loop to intelligently adjust its image acquisition parameters. The method involves analyzing a first captured image to identify a feature of interest and assess its characteristics, and then using this information to optimize the parameters for a second, subsequent image capture to enhance the visibility of the identified feature.

### BACKGROUND

People with diabetes commonly suffer from a condition known as diabetic foot ulcers (DFU). It is recommended that diabetics inspect their feet daily to detect any abnormal damage to the skin that may be an indicator of the onset of DFU. However, factors such as reduced vision and mobility make this difficult. To address these limitations, automated skin inspection systems have been developed. These systems are advantageous as they can inspect for abnormalities using multiple types of data, such as a combination of temperature and visual data, which provides more information than any single sensing modality.

For such automated skin inspection systems to be diagnostically effective, they must be capable of capturing high-quality images that clearly reveal potential abnormalities. A fundamental challenge in these systems, however, lies in the image acquisition process itself. Typically, these devices operate using a fixed, predefined set of image acquisition parameters, such as exposure time, illumination intensity, and contrast. While these static settings may be suitable for capturing a general overview of the inspection area, they represent a compromise and are often suboptimal for visualizing specific features of clinical interest.

For example, acquisition parameters set for a well-lit, life-like image may fail to capture sufficient detail in a dark or low-contrast feature, such as a small lesion or the subtle texture of dry skin. Conversely, if the parameters are adjusted to be bright enough to reveal such a feature, the rest of the image may become overexposed, washing out other important details. This static, “one-size-fits-all” imaging approach lacks the ability to intelligently respond to the specific content of the image being captured. Once an initial image is taken, the opportunity to get a better, more targeted view of a potential problem area is lost.

Therefore, a need exists for a more intelligent and dynamic imaging method.

### SUMMARY

The present disclosure provides a method and system for operating a skin inspection device that overcomes the limitations of static imaging systems. The disclosure is directed to a method of adaptive image acquisition, wherein the device intelligently analyzes an initial image and dynamically adjusts its own settings to capture a second, optimized image of a specific feature of interest, thereby significantly enhancing its diagnostic capability.

In one aspect, a method for operating a skin inspection device is provided. The method comprises capturing a first image of an inspection area and analyzing it with a processor to identify a feature of interest. Based on one or more characteristics of this feature, such as its location, size, color, or an assessed risk level, the processor determines a set of adjusted image acquisition parameters. A second, feature inspection image is then captured using these adjusted parameters, which are specifically configured to optimize the visibility of the identified feature.

In further aspects, the method may include adjusting parameters such as illumination intensity, exposure time, or contrast. The method may also involve capturing multiple images with different settings and combining them to create a high dynamic range (HDR) image. The acquisition parameters may also be configured based on the intended purpose of the image, such as providing a life-like appearance for general visual inspection or minimizing color signal noise for temperature measurement.

In another aspect, a skin inspection device configured to perform this method is disclosed. The device comprises an image capture device, an illumination source, and a processor operatively coupled to them. The processor is configured to control the capture of the first and second images and to determine the adjusted acquisition parameters based on its analysis. The device may operate as part of a larger system, receiving commands via a feedback loop from a remote data monitoring system based on a determined risk level. The device may also be configured to perform pre-scan checks, identify the user, and include advanced hardware features such as a knife-edge aperture for glare reduction.

In yet another aspect, a skin inspection system is provided, comprising an image capture device, an illumination source, a processor, and a memory storing instructions that, when executed, cause the system to perform the adaptive imaging method. The system may also include a temperature sensor array, allowing the processor to analyze both visual and thermal data.

The disclosure also provides for a method of assessing abnormality risk, wherein an alert threshold for a first patient metric is dynamically adjusted based on a change in a second patient metric. Additionally, a non-transitory computer-readable medium storing instructions for performing the adaptive imaging method is disclosed, as is a method for clinically reviewing a skin condition that involves the remote command and control of the skin inspection device.

These and other aspects of the disclosure will be better understood with reference to the followings Figures which are provided to assist in an understanding of the present teaching.

### BRIEF DESCRIPTION OF THE DRAWINGS

The present teaching will now be described with reference to the accompanying drawings in which:

FIG. 1 illustrates a skin abnormality detection system.

FIG. 2 illustrates a skin inspection device.

FIG. 3 illustrates a cross-sectional view of an exemplary skin inspection device.

FIG. 4 illustrates exemplary optics.

FIG. 5 illustrates an exemplary skin inspection device.

FIG. 6 illustrates exemplary distortion of an object through a wide-angle lens.

FIG. 7 illustrates the impact of distortion to a rectilinear sensor array when observed through a wide-angle lens.

FIG. 8 illustrates that the position of the field of view of the lens can vary within the digital image.

FIG. 9 illustrates how the position of a sensor may be specified by its central pixel coordinate in a digital image.

FIG. 10 illustrates a flowchart teaching how a map can be created to link an indexed array of sensors to their positions in a digital image.

FIG. 11 illustrates a flowchart of an additional embodiment teaching how a map can be created to link an indexed array of sensors to their positions in a digital image.

FIG. 12 illustrates an exemplary matching algorithm.

FIG. 13 illustrates the outputs of a detection and indexing process.

FIG. 14 illustrates a scan inspection pane of a user interface, used for reviewing scan for abnormalities, including inspecting thermal and visual data.

FIG. 15 illustrates a patient profile view of a user interface including display of various metrics and history.

FIG. 16 illustrates the functionality of a user interface to determine the nearest sensor to the position of a pointer on a visual image by using a map.

FIG. 17 illustrated a population metrics view of a user interface.

FIG. 18 illustrates a user interface for annotating scan data.

FIG. 19 illustrated performing feature analysis in time series data.

FIG. 20 illustrates a skin inspection device having image capture devices and light sources for capturing images of the sides and upper regions of the feet.

FIG. 21 illustrated mapping an indexed sensor array with corresponding positions in an image.

FIG. 22 illustrates a flow chart of a multi-exposure, multi-sample ROI image analysis chain.

FIG. 23 illustrates a de-warping of an image captured from a wide-angle lens using a grid array of known geometry.

FIG. 24 illustrates a flow chart of the scan capture sequence of a skin inspection device

FIG. 25 illustrates how the internal surfaces and position of features of a skin inspection device can impact indirect glare.

FIG. 26 illustrates how the geometry of a light source aperture can impact image artefacts.

FIG. 27 illustrates the surface finish of internal structures of a skin inspection device can reduce ambient light noise.

FIG. 28 illustrates a flowchart for a remote monitoring workflow that uses multimodal processing to generate clinical risks and context for determining a clinical action.

FIG. 29 illustrates a flowchart for a risk assessment method based on comparing the observed characteristics of a detected feature with predicted characteristics derived from longitudinal observations.

### DETAILED DESCRIPTION OF THE DRAWINGS

The present disclosure will now be described with reference to some exemplary skin inspection devices and user interfaces. It will be understood that the exemplary skin inspection devices and user interfaces are provided to assist in an understanding of the teaching and is not to be construed as limiting in any fashion. Furthermore, elements or components that are described with reference to any one Figure may be interchanged with those of other Figures or other equivalent elements without departing from the spirit of the present teaching. It will be appreciated that for simplicity and clarity of illustration, where considered appropriate, reference numerals may be repeated among the figures to indicate corresponding or analogous elements.

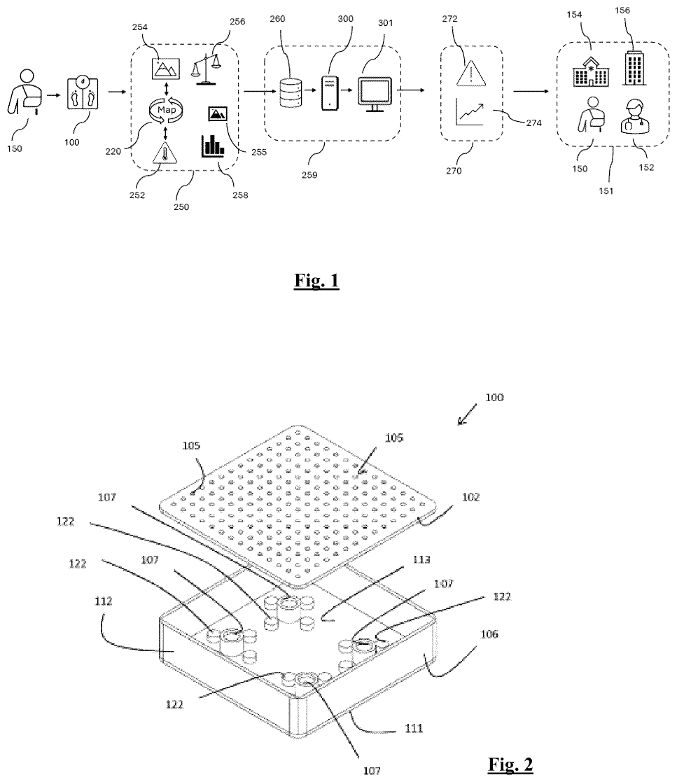

A skin abnormality detection system is illustrated in FIG. 1. A patient user 150 is provided a skin inspection device 100. Data is recorded by the skin inspection device 100 when the patient user 100 stands on it. The data recorded may include visual image data 254, feature detection images 255, temperature data 252, weight data 256, and metadata 258. Metadata 258 may include information such as the time the scan was taken, ambient temperature, device metrics such as level of cellular connectivity, time since the device was powered on, time stamped events such as processing time, network events, user interactions, unique device or file identifiers such as registration numbers, public keys and file UUIDS and so on. The scan also includes a map 220, which acts as a transfer function. This map 220 provides the crucial link between the datasets by correlating the discrete temperature data 252 with the spatial coordinates of both the visual inspection images 254 and the feature detection images 255, allowing all datasets to be analyzed concurrently.

The data recorded may be packaged as a scan 250. The scan 250 may be compressed to reduce file transfer size, which reduces the time required to transmit a scan. It also allows the scan 250 to be transmitted over lower bandwidth communication networks. The scan 250 may be encrypted prior to transmission to increase the level of security of the file transfer process and reduces the likelihood of disclosure of the data.

The scan 250 is transmitted to a data monitoring system 259. The data monitoring system 259 may be local i.e., part of the skin inspection device 100 or it may be a remote system. The scan 250 is received into a database 260. The database contains further data and information that can be used to determine abnormalities such as patient medical history, historical scan data, patient information and behavioural information. The data in the database and the scan 250 are inspected for abnormalities by a computer 300, and or by a person using a graphical user interface 301.

If an abnormality is detected during the review of the scan 250 a communication 270 may be provided, automatically or manually, which may contain an abnormality alert 272, and/or abnormality data 274. The communication may be generated and sent by a processor, or by a person. The communication 270 may be shared to the care team 151 which may comprise the patient user 150, the healthcare professional 152, the provider 154, or the payor 156. The care team 151 can interpret the communication 270 and take action to prevention the potential abnormality detected developing into a more serious issue.

Referring now to FIG. 2 which illustrates the skin inspection device 100 for identifying the formation of abnormalities in accordance with the present teaching. The device 100 comprises a transparent panel 102 which defines an inspection area for co-operating with a region of a body under inspection. For example, the region under inspection may be a foot, a hand, an arm, a leg, etc. In the exemplary arrangement, the region under inspection is a sole of a foot 105. A temperature sensor is located near to or on the upper surface of the transparent panel 102. A rectilinear array of temperature sensors 105 is provided in detect temperature data over a 2-dimensional area.

Preferably, the panel 102 may be supported on a housing 106 which may accommodate the components of the skin inspection device 100 via a hollow interior region 113. Typically, the housing 106 comprises a base 111 with sidewalls 112 which extend upwardly therefrom, defining the hollow interior region 113. Typically, within the hollow interior region 113 are image capture devices (for example, cameras) 107 for capturing an image of the temperature sensors 105 and the foot in contact with the panel 102. At least one light source 122 is also optionally provided within the hollow interior region 113. The light sources may be LEDs, cathode lamps, electroluminescent coated materials and the like. Optionally, a CPU (not shown) may also be located in the hollow interior region 113 and is configured to control the operations of the device.

In some embodiments, the temperature sensors 105 may be provided on the panel 102 as printed, flexible electronic, or optical components. They may be printed directly onto the panel 102 or alternatively, printed onto a transparent overlay film such as Polyester or Polyethylene terephthalate glycol (PETG), which may be called an interlayer 110. The interlayer 110 may have a thickness of around 0.1 mm, for example. This may be subsequently attached to the transparent panel. However, it will be appreciated by those skilled in the art that the temperature sensors 105 may be provided on the panel 102 or interlayer 110 by other suitable means such as adhering using glue, bonding agents, or other adhesives. The temperature sensors 105 may also be fixed to the transparent panel 102 using other methods such as mechanical fixings, laminating in position using a film, and so on.

The temperature sensors 105 may be any suitable kind, such as, but not limited to, contact/non-contact sensors, Resistance Temperature Detectors (RTDs), thermocouples, thermopiles, thermistors, semiconductors, microbolometers, where an electrical property (voltage, current, resistance etc) changes with a change in temperature. Alternatively, materials such as thermochromic liquid crystals (TLCs) may be used, where a visible property (hue, saturation, value etc) changes with a change in temperature.

In preferred embodiments, the temperature sensors 105 are provided on the upper side of the interlayer 110 which is on the upper side of the transparent panel 102. This allows that the sensors 105 easily contact the region of the body under inspection (e.g., the sole of the foot). Preferably, the sensors 105 are positioned such that the image capture devices 107 are provided with maximum visibility through the transparent panel 102. The sensors may be connected via connection wires or traces. Preferably, the sensors 105 and connection wires are arranged to provide maximum visibility through the panel 102 to the image capture devices 107.

For TLC sensors, the change in visible property corresponding to a change in temperature may be detected optically and hence connection wires or traces are not required. Preferably, the sensors 105 are designed to provide maximum visibility through the transparent panel 102 to the image capture devices 107.

In an exemplary embodiment, the sensors 105 are arranged in a grid with a pitch in a range of about 0.5 cm-2 cm, to provide adequate resolution to record the skin temperature. It is not intended to limit the present disclosure to the exemplary grid configuration described herein as a grid with alternative pitch ranges is also envisaged.

In preferred embodiments, panel 102 has sufficient strength to support the weight of an adult human.

Further, as the foot has various contours, for example the arch, the entire sole of the foot may not be in contact with the temperature sensors 105. In order to improve the contact between the temperature sensors 105 and the foot, the panel 102 or interlayer 110 or both may be manufactured from a flexible or resilient material that would conform to the shape of the sole of the foot. A material such as clear silicone may be used as it is both optically transparent and resilient. For example, the panel may conform to match the shape of the arch of the user's foot. This would allow more contact with the temperature sensors.

In an exemplary arrangement, the panel may include one or more formations for engaging with the foot in order to enhance the area of the foot that is in contact with the temperature sensors 105. For example, the one or more formations may include one or more indentations or one or more projections or a combination of indentations and projections. It is not intended to limit the present teaching to silicone as other materials with similar properties may be used as would be understood by those skilled in the art. The temperature sensors 105 may be printed onto this layer in the same fashion as outlined above.

FIG. 3 illustrates a cross-sectional view of an exemplary skin inspection device, showcasing the various components integral to its function in inspecting a patient's foot for abnormalities, such as diabetic foot ulcers. The patient's foot 101, representing the region of the body under inspection, is placed in contact with the device. A position guide 126 ensures consistent placement of the foot on different days, which minimizes variation and improves the accuracy and reliability of the captured data.

The foot makes contact with an array of temperature sensors 105 that detect temperature changes. In this embodiment, these sensors are provided on a transparent interlayer 110, such as an overlay film, which is positioned on the upper surface of the transparent panel 102. The transparent panel 102 defines the inspection area, allows light to pass through for clear images, and may be made from a flexible material like clear silicone to conform to the shape of the foot and ensure adequate contact with the sensors.

The entire assembly is supported by a housing 106, which is comprised of sidewalls 112 and a base, defining the hollow interior 113. This interior is shielded from external light to ensure accurate image capture. Positioned within this shielded interior is the centrally located image capture device 107, typically a camera, which includes a lens 121 to focus incoming light and capture high-quality images. Also within the interior are light sources 122, such as LEDs, which provide clear and uniform illumination for the foot, enhancing the visibility of skin features.

This configuration enables the simultaneous capture of thermal and visual data. The position guide ensures consistent foot placement, the transparent panel and sensors facilitate data collection, and the internal optics and lighting ensure high-quality imaging for comprehensive and efficient inspection.

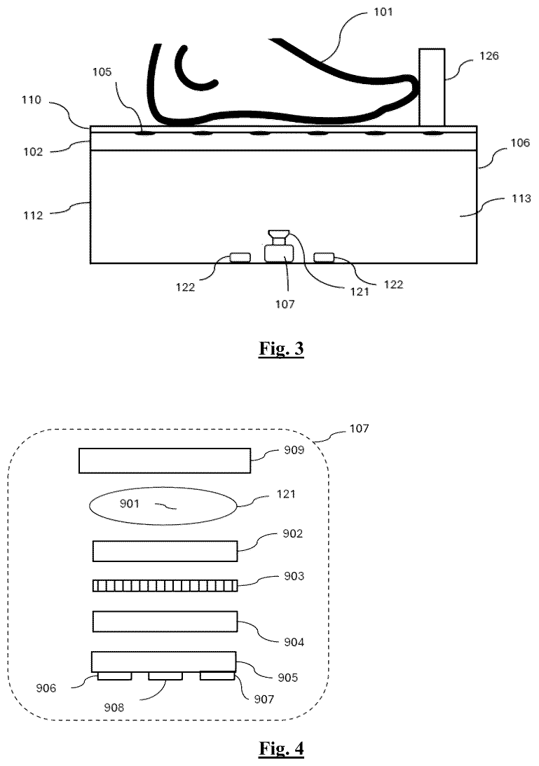

FIG. 4 illustrates the exemplary optical and electronic components within an image capture device 107 used for identifying skin abnormalities. This setup is designed to capture high-quality images, allowing for detailed inspection. The optical path begins with a lens 121, which focuses incoming light. The lens is held in place by a lens mount 909 that provides mechanical stability and ensures precise alignment. To enhance image clarity, an anti-reflection coating (ARC) 901 may be applied to the lens 121 or other transparent surfaces to minimize glare. A filter 902, such as a neutral density, infrared, or polarizing filter, can be used to optimize the light conditions for image capture.

The focused and filtered light then passes through a color filter Array (CFA) 903, which allows the sensor to capture color information by filtering light into red, green, and blue channels. The light ultimately reaches the image sensor 904 (e.g., a CMOS or CCD sensor), which converts the light into an electrical signal to form a digital image.

The electronic components are housed on a printed circuit board (PCB) 905. This board includes integrated circuit (IC) components 906, such as processors and memory chips that control device operations. Input/Output (I/O) connectors 907 facilitate data transfer and device control with external systems, while signal processing components 908 enhance image quality and extract relevant data from the sensor signal.

The integration of these various filters, coatings, and processing components ensures that the captured images are clear and accurately represent the visual and thermal state of the skin.

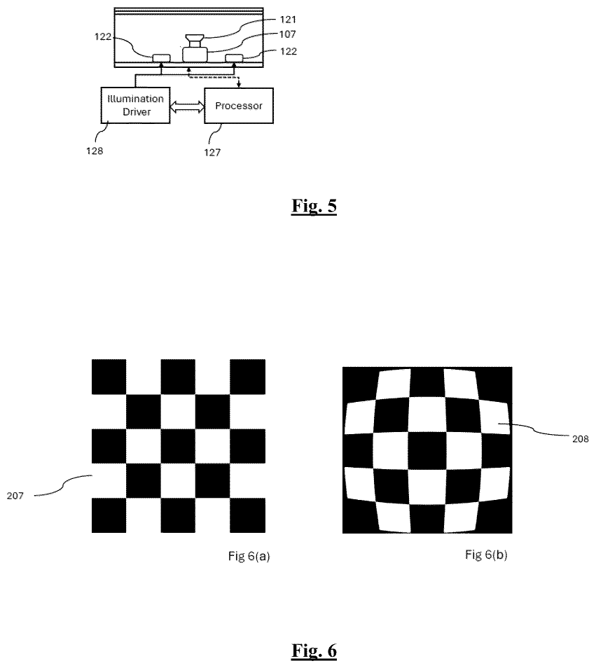

FIG. 5 shows the image capture arrangement designed to optimize the capture of high-quality images for the skin inspection device. The system is controlled by a central processor 127, which communicates with an illumination driver 128. The driver 128 controls the illumination sources 122 to provide stable and uniform illumination, which minimizes shadows and glare.

Light from the sources is focused by a lens 121 onto the image sensor within the image capture device 107, where it is converted into a digital image. The processor 127 also programs the image sensor and processes the captured images, applying various adjustments to ensure they accurately represent the inspection area. This configuration allows for detailed visual inspections, facilitating the identification of skin abnormalities such as diabetic foot ulcers.

FIG. 6 illustrates the image warping effect, known as barrel distortion, that occurs when an object is observed through a wide-angle lens. FIG. 6(a) shows an exemplary target 207, which is a rectilinear checkerboard pattern with straight lines. This represents the object as it appears without distortion. FIG. 6(b) depicts the resulting image of the target with distortion 208 as captured through a wide-angle lens. This image demonstrates the significant geometric distortion, which is characterized by the curvature of straight lines, an effect that is especially noticeable towards the edges of the image.

FIG. 6 highlights how a wide-angle lens can bend straight lines and compress distances towards the periphery of the image. Understanding and correcting for this barrel distortion is essential when designing optical systems for precise measurements, such as those used in skin inspection devices.

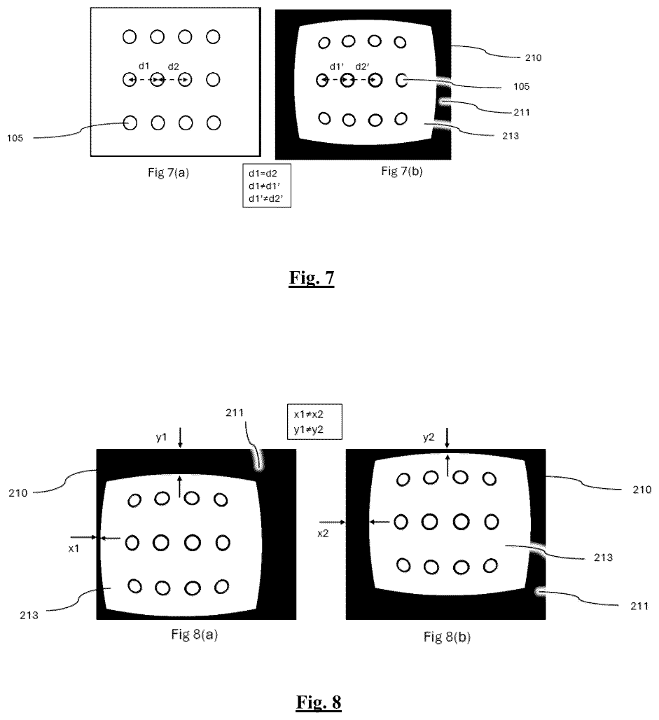

FIG. 7 illustrates how a wide-angle lens distorts the perceived geometry of a sensor array. FIG. 7(a) shows the physical layout of a rectilinear sensor array 105. In this undistorted view, the distances between adjacent sensors 105 are equal, as indicated by the relationship d1=d2. FIG. 7(b) shows the digital image 210 of the same sensor array as captured by a wide-angle lens. The image demonstrates significant barrel distortion, causing the sensor array 105 to appear compressed, particularly towards the edges. This distortion alters the perceived spatial relationship between the sensors. The previously equal distances are now unequal, as shown by d1′≠d2′. The active image area containing the sensor array is also referred to as the field of view 213, which is surrounded by an unused area of the sensor, or deadspace 211.

This figure emphasizes the non-uniform distortion introduced by a wide-angle lens, which affects the spatial relationship between sensors and necessitates correction techniques in data analysis.

FIG. 8 illustrates that the position of the field of view 213 can vary within the digital image 210 captured from a wide-angle lens. This variation can be caused by minor differences in lens characteristics or camera positioning during manufacturing. The digital image 210 refers to the entire captured frame, which includes both the active image area, known as the field of view 213, and the surrounding black, unused area, known as deadspace 211.

The figure demonstrates this positional variation by comparing two separate captures. In FIG. 8(a), the field of view 213 is shown in one position, with its location defined by the horizontal distance x1 and vertical distance y1 from the image edges. In FIG. 8(b), the field of view 213 has shifted to a different position, now defined by the distances x2 and y2. The inequality relationships, x1≠x2 and y1≠y2, explicitly confirm this shift.

In summary, FIG. 8 highlights a manufacturing and calibration challenge: the exact position of the useful image data can vary between devices, necessitating an automated method for locating and indexing features within the field of view.

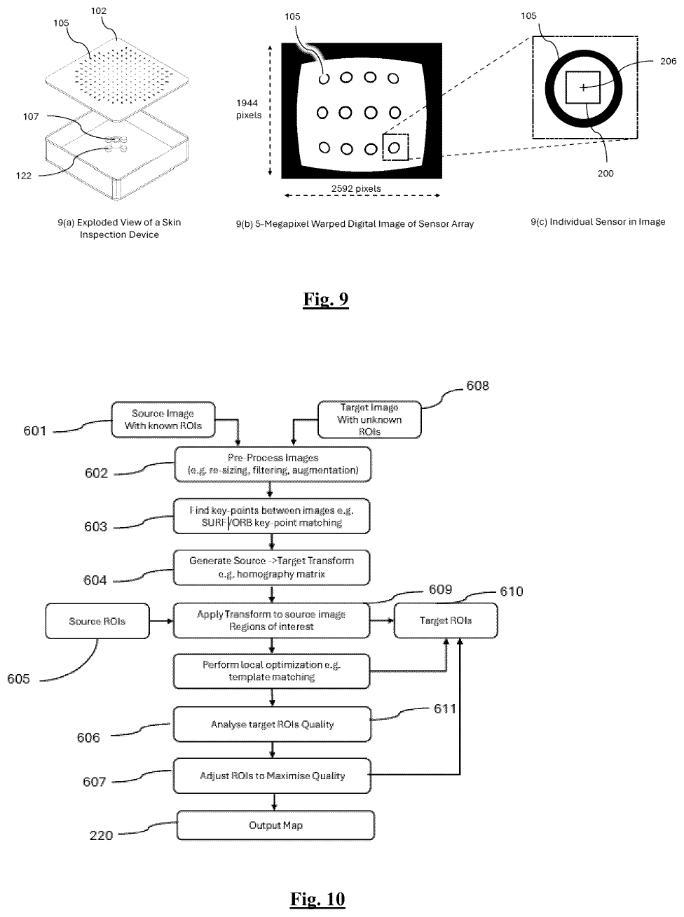

FIG. 9 illustrates the relationship between the physical sensor array and its representation in a digital image, a key aspect of sensor position detection. FIG. 9(a) shows an exploded view of the skin inspection device. It features a transparent panel 102 on which an array of sensors 105 are mounted. Below this panel is the housing containing the image capture device 107 and light sources 122.

FIG. 9(b) represents a 5-Megapixel (2592×1944 pixels) warped digital image of the sensor array as captured by the image capture device 107. The physical array of sensors 105 appears distorted in this image due to the wide-angle lens. FIG. 9(c) provides a magnified, detailed view of an individual sensor as it appears in the digital image. This view defines how each sensor is located and analyzed.

The sensor 105 itself is identified, and a specific sensor region of interest (ROI) 200 is defined around it for data extraction. The precise location of the sensor within the image is specified by its sensor centre pixel coordinate 206. This combination of identifying the ROI and its central pixel coordinate is crucial for accurate data extraction and analysis from the warped image.

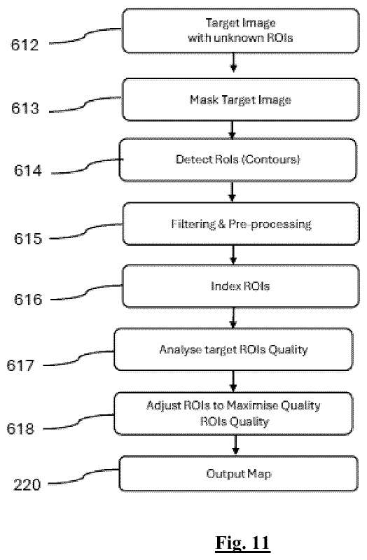

FIG. 10 illustrates a flowchart for the sensor registration process, which maps sensor data from a source image to a target image. This systematic approach is crucial for applications like skin inspection devices, where accurate alignment of thermal and visual data is necessary.

The process begins with two inputs: a source image with known ROIs 601, which serves as a reference, and a target image with unknown ROIs 608, which is the image to be analyzed. Both images undergo pre-processing 602, which may include resizing, filtering, or augmentation to facilitate matching.

Next, the system proceeds to find key-points between images 603 using algorithms like SURF or ORB to identify common features. Based on these matched key-points, the system will generate a source-to-target transform 604, such as a homography matrix, that maps coordinates from the source to the target image.

This transform is then used to apply the transform to the source image regions of interest 609. This step takes the known source ROIs 605 and projects them onto the target image to determine the initial positions of the target ROIs 610.

To refine these initial positions, the system will perform local optimization 606, for example, using template matching. The quality of the resulting target ROIs is then checked in the analyse target ROIs quality, step 611. Based on this analysis, the system will adjust ROIs to maximise quality 607, which may involve moving the ROI bounding box to improve the quality metrics. This optimized position is then fed back to the target ROIs 610.

Once the ROIs are accurately positioned and optimized, a final output map 220 is created. This map links the sensor coordinates in the target image with an index, enabling the simultaneous and correlated evaluation of different data types.

FIG. 11 illustrates a flowchart detailing a systematic approach for contour detection indexing, which is an alternative method for mapping sensor data in a target image. This process is essential for applications like skin inspection devices, where precise alignment of thermal and visual data is required for identifying abnormalities such as diabetic foot ulcers.

The process begins with a target image with unknown ROIs 612 as its input. First, the system will mask the target image 613, a step which highlights the sensor locations to simplify their identification. Using this masked image, the system proceeds to detect ROIs contours 614, identifying the shapes and locations of the sensors using computer vision techniques.

The detected contours then undergo filtering and pre-processing 615. This step ensures the contours meet predefined thresholds for size and location and may also compensate for lens distortion. Following this, the system will index the ROIs 616 by mapping the detected contours against a known indexing pattern, such as a grid. Once indexed, the quality of the ROIs is evaluated in the analyse target ROIs quality, step 617. Based on this analysis, the system will adjust the ROIs to maximise quality 618, which may involve moving the ROI bounding box to a position with better quality metrics.

Finally, after the ROIs have been accurately indexed and optimized, an output map 220 is created. This map links the final sensor coordinates to their respective indices, ensuring that sensor positions are accurately determined and that data from different domains can be reliably correlated.

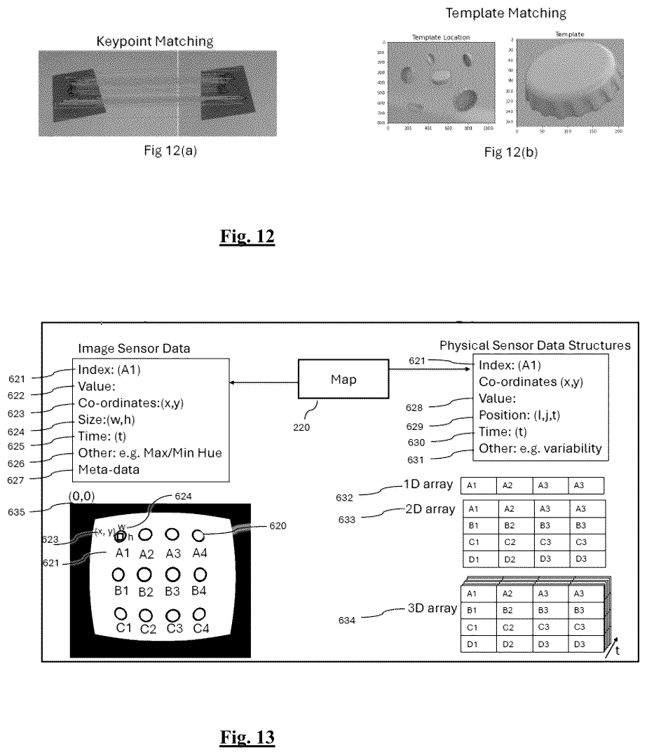

FIG. 12(a) shows an example of keypoint matching. This process involves applying key-point detection algorithms (such as SIFT, ORB, or SURF) to identify distinct and identifiable features—like corners or blobs—in both a source image and a target image. These key-points serve as reference points for alignment. As shown, lines are drawn between the two images to indicate successful matches between corresponding key-points.

FIG. 12(b) shows an example of template matching. In this process, a smaller image of a known feature (the “Template”) is used to find its location within a larger image (the “Template Location”). The algorithm searches the larger image to find the area that best matches the template.

The key-point detection and matching process shown in FIG. 12(a) is crucial for generating a homography matrix. This matrix is a transformation that maps the coordinates of the key-points in the source image to their corresponding positions in the target image, accounting for geometric distortions and enabling accurate alignment of the regions of interest (ROIs). This method is particularly valuable in applications like skin inspection devices, where the accurate alignment of visual and thermal data is essential for identifying abnormalities.

FIG. 13 illustrates the output of the detection and indexing process, showcasing how various data elements from image sensors and physical sensors are captured, indexed, and mapped to enable simultaneous analysis. The process links two main types of data via a map 220, which acts as a transfer function. First, the image sensor data is captured from the image 620. For each sensor identified in the image, the following data is recorded:
An Index 621, such as (A1), which is a unique identifier for the sensor's position.
A Value 622, which is the measured parameter from the Region of Interest (ROI), such as a mean hue value.
The Coordinates 623, which are the (x,y) pixel coordinates describing the center of the ROI relative to an origin point 635.
The Size 624 of the ROI, given as width (w) and height (h).
The Time 625 of data capture.
Other 626 parameters calculated from the image, such as Max/Min Hue.
Meta-data 627, including supplementary information like sensor type or lighting configuration.

This image sensor data is then mapped to the physical sensor data structures. This structure contains corresponding information for each physical sensor:
An Index 621 that matches the index from the image data.
Coordinates and a Value 628, which is the direct reading from the physical sensor (e.g., temperature).
The physical Position 629 of the sensor within its array structure, using indices for rows (i), columns (j), and time (t).
The Time 630 of data capture.
Other 631 relevant information, such as the variability of sensor readings.

The physical sensor data can be organized into various structures, such as a 1D array 632 for a linear arrangement, a 2D array 633 for a grid-like arrangement, or a 3D array 634 which captures data over time or in layers. This comprehensive mapping process ensures that all data elements are captured, indexed, and correlated, enabling a robust and simultaneous evaluation of visual and temperature data for identifying skin abnormalities.

FIG. 14 illustrates a scan inspection pane 309, which is a graphical user interface (GUI) for the simultaneous inspection of thermal and visual data from human feet, aiding in the detection of abnormalities like diabetic foot ulcers. The interface is organized with a series of tabs 310, such as “Inspect,” “History,” and “Annotate,” which allow users to navigate between different functionalities. The main inspection area is the visual inspection pane 312, which shows stitched and horizontally aligned images of the patient's feet. This pane can display a de-warped image of the right foot 303 for a corrected view and a warped image of the left foot 304 showing the raw, wide-angle capture. The array of temperature sensors 201 is visible as small dots on the images, with the entire sensor array 105 also indicated.

A user can interact with the pane using a pointer 302 to highlight a selected sensor 306. The temperature of this selected sensor 307 is then populated into the temperature asymmetry table 305. This table displays and compares temperature readings from corresponding locations on the right and left feet to identify potential asymmetries. The interface also includes several control panels. A set of observation and action radio buttons 314 allows users to log their findings. Heatmap controls 320 enable users to toggle a thermal heatmap overlay, adjust its transparency, and select a color scale.

Finally, scan toggle buttons 324 allow users to navigate between different scans for comparison over time. Additional toggles to enable features such as de-speckling, contrast enhancement, background removal, image sharpening, brightness, colour correction or normalization

This detailed description of FIG. 14 provides an in-depth understanding of the components and their interconnections, highlighting the innovative aspects of the system for simultaneous thermal and visual data inspection.

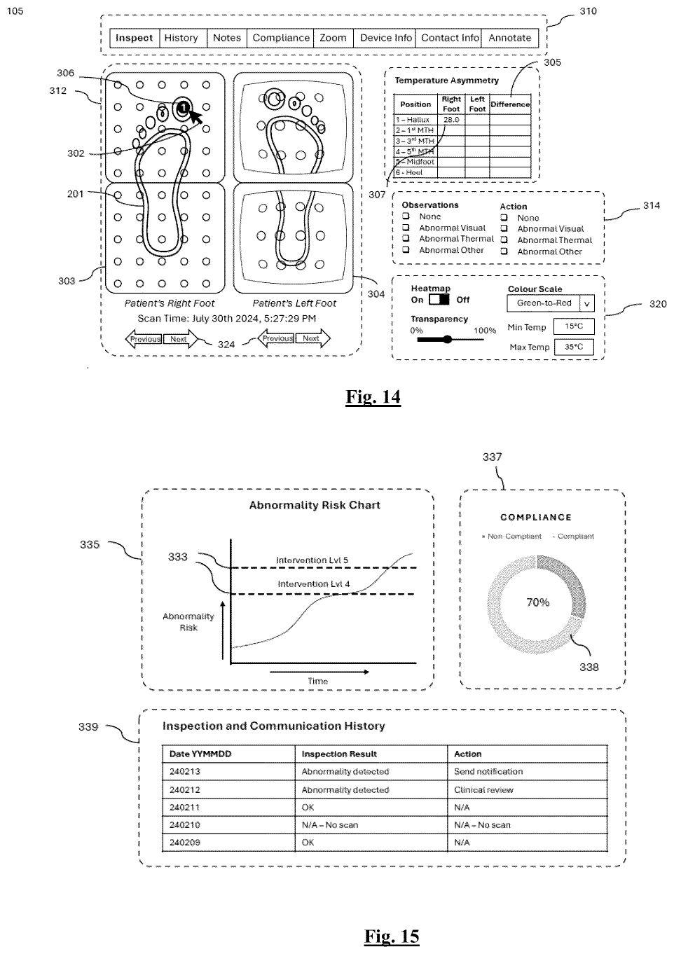

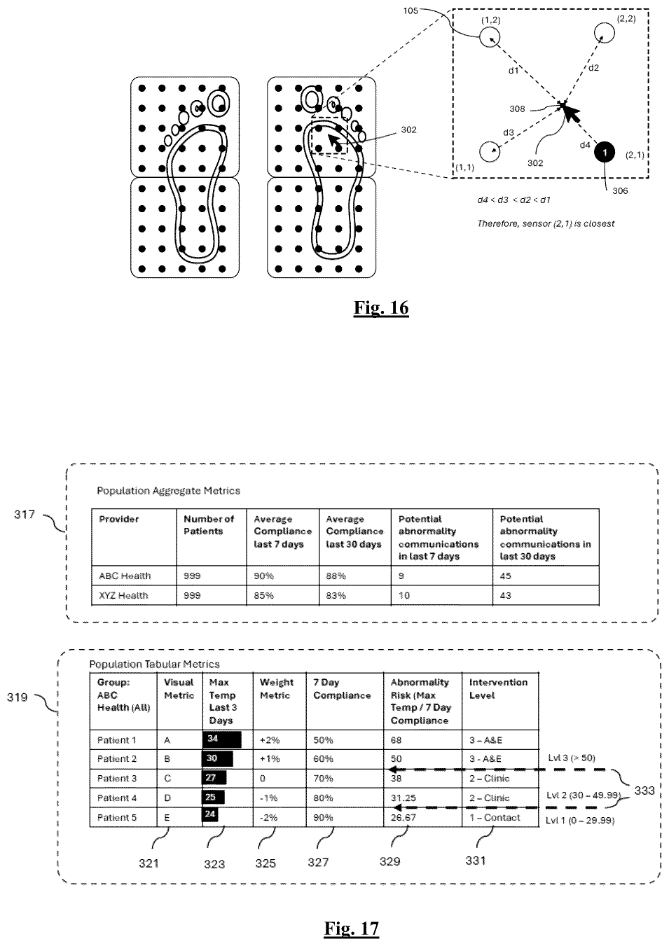

FIG. 15 illustrates a patient profile interface within a skin inspection system, which is used for tracking and managing the risk of skin abnormalities such as diabetic foot ulcers. This interface provides a comprehensive view of a patient's risk, compliance, and history.

The abnormality risk chart section 335 contains a graphical representation of the patient's abnormality risk over time. The risk chart itself 333 plots risk (y-axis) against time (x-axis) and includes various intervention levels, such as Level 4 and Level 5. These levels indicate thresholds at which specific actions are triggered based on the patient's risk score.

The compliance section 337 provides an overview of the patient's adherence to inspection protocols. It features a donut chart 338 that visually represents the proportion of compliant and non-compliant inspections, with the specific compliance percentage shown in the center (e.g., 70%).

The inspection and communication history table 339 records the dates, results, and actions taken for past inspections. Each row corresponds to a specific inspection date, detailing whether an abnormality was detected and the subsequent action taken, such as sending a notification or prompting a clinical review. The row may also include a link to a detailed escalation report outlining the issue and presenting all relevant contextual data and images

This detailed description of FIG. 15 provides an in-depth understanding of the components and their interconnections within the patient profile interface, highlighting the innovative aspects of the present disclosure for managing and monitoring skin abnormalities.

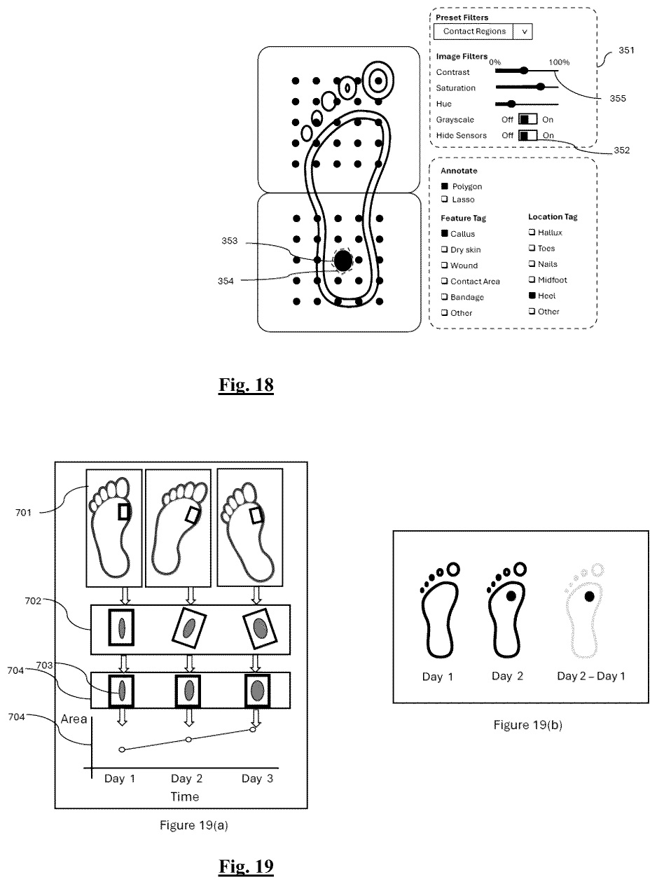

FIG. 16 illustrates a graphical user interface (GUI) that detects the nearest sensor to the cursor, a feature designed to help users efficiently correlate visual and thermal data during skin inspections. The visual inspection panes show the array of temperature sensors 105 embedded in the device as a grid of small dots. The enlarged section demonstrates the process that occurs when a user clicks a specific point on the visual image with the pointer 302. The system identifies the selected pixel coordinate 308, which serves as the reference point for calculating distances to the nearby sensors.

The system then calculates the distances (d1, d2, d3, and d4) from this selected coordinate 308 to the four adjacent sensors, which in this example are indexed as (1,1), (1,2), (2,2), and (2,1). The nearest sensor 306 is identified based on the shortest calculated distance. As shown, the distance d4 is the shortest, making sensor (2,1) the closest sensor. This nearest sensor 306 is then highlighted within the visual inspection pane, allowing the user to quickly and accurately select the relevant temperature data for the area of interest.

This detailed description of FIG. 16 provides an in-depth understanding of the components and their function, highlighting the innovative aspects of the GUI for efficient data correlation.

FIG. 17 illustrates a population management user interface designed for monitoring and managing the risk of skin abnormalities across a population of patients. This interface enables healthcare professionals to efficiently track compliance and identify potential issues by providing both aggregated and detailed patient metrics.

The top section of the interface displays population aggregate metrics 317, which provides a summary of key metrics aggregated at the provider level. This table includes columns for the provider's name, the number of patients being monitored, and the average compliance rates for the last 7 and 30 days, as well as the number of potential abnormality communications sent during those periods.

The main part of the interface features population tabular metrics 319, which provides detailed data for individual patients within a selected group. For each patient, the table includes:
A Visual Metric 321, which is a grade (e.g., A, B, C) that categorizes the visual condition of the patient's feet.
The Max Temp Last 3 Days 323, showing the maximum temperature recorded.
A Weight Metric 325, indicating the patient's percentage weight change.
The 7 Day Compliance 327 rate.
A calculated Abnormality Risk 329 score, which in this example is based on the maximum temperature divided by the 7-day compliance rate.
The resulting Intervention Level 331, such as “A&E,” “Clinic,” or “Contact.”

The intervention level is determined by a set of intervention thresholds 333, which define the action required based on the abnormality risk score. For example, a score greater than 50 may trigger a Level 3 (A&E) intervention, while a score between 30 and 49.99 triggers a Level 2 (Clinic) intervention.

This detailed description of FIG. 17 provides an in-depth understanding of the components and their interconnections, highlighting the innovative aspects of the population management user interface for efficiently managing and monitoring skin abnormalities.

FIG. 18 illustrates the annotation UI, a graphical user interface (GUI) within the skin inspection system that allows users to annotate and analyze visual data, facilitating the identification and tracking of skin abnormalities.

The UI includes a filter menu 351 which provides various options for adjusting the visual properties of the image. Users can apply different image filters 355, such as sliders for contrast, saturation, and hue, or toggle a greyscale view. This menu also includes a hide sensor toggle 352, which allows users to hide or display the temperature sensors in the visual image to make it easier to inspect the underlying skin. Additionally, users can apply preset filters, such as “Contact Regions,” for optimized viewing of specific features.

The pane also provides a set of annotation tools 353 for highlighting and tagging features of interest. These annotations may be created manually, or using computer vision algorithms or machine learning models. Polygons or lassos, or pixel masks may be generated to create a precise boundary around a feature. They can then apply classification tags, such as a “Feature Tag” (e.g., Callus, Wound, Bandage, Clothing, Soiling, Poor Positioning) and a “Location Tag” (e.g., hallux, second toe, little toes, medial forefoot, central forefoot, lateral forefoot, medial midfoot, lateral midfoot, medial heel, and lateral heel). These tags can then be used to generate or tune clinical risk profiles, automate clinical report generation, drive clinical follow up.

While not explicitly numbered in this figure, the pane also supports measurement tools 354, which enable users to measure various metrics related to the annotated features, such as their size, shape, and color. Furthermore, the system can maintain an annotation history 355, which logs all annotations made, including timestamps and user details.

This detailed description of FIG. 18 provides an in-depth understanding of the components and their interconnections within the Annotation Pane, highlighting the innovative aspects of the present disclosure for efficiently annotating and analyzing visual data.

FIG. 19 illustrates the process of feature analysis in time series data, which is used for detecting and tracking skin abnormalities over multiple days. FIG. 19(a) demonstrates the feature analysis process over a three-day period. The process begins with the initial feature detection 701, where a potential abnormality, such as a callus, is identified and highlighted on the foot images for Day 1, Day 2, and Day 3. This is followed by image pre-processing and cropping 702, where the highlighted feature is isolated from the full foot image to allow for a more detailed inspection. The isolated features then undergo alignment and tracking 703 to ensure they have a consistent position and orientation across all days, which facilitates comparison.

Once aligned, the isolated feature is further processed to measure specific metrics, such as its area, width, size, shape, color, and texture. The change in this metric over time is then plotted in the temporal analysis graph, which shows the area 704 on the y-axis against time on the x-axis. This graph provides insights into whether the abnormality is growing, shrinking, or remaining stable.

FIG. 19(b) illustrates an alternative method for change detection using image subtraction. It shows images of a feature on Day 1 and Day 2, allowing for direct visual comparison. The “Day 2-Day 1 Image” represents the result of subtracting the Day 1 image from the Day 2 image. This subtraction process highlights the differences between the two days, making it easier to identify any changes in the feature's appearance, such as growth or reduction in size.

This detailed description of FIG. 19 provides an in-depth understanding of the feature analysis process, highlighting the innovative aspects of the present disclosure for efficiently detecting, tracking, and analyzing skin abnormalities over time.

FIG. 20 illustrates a skin inspection device 100 designed for the comprehensive inspection of human feet, including areas that are typically difficult to visualize. This device integrates multiple imaging components to capture both thermal and visual data for identifying abnormalities such as diabetic foot ulcers. Embedded within the transparent panel of the device is an array of temperature sensors 105, which are responsible for detecting temperature variations across the soles of the feet.

To provide a comprehensive visual record, multiple image capture devices 107, typically cameras, are positioned around the device to capture high-resolution images from various angles. These cameras work in conjunction with strategically placed light sources 122, such as LEDs, which ensure that the captured images are clear and well-lit.

To visualize hard-to-reach areas like the sides and tops of the toes and feet, the device incorporates additional optical components. Concave mirrors 124 are positioned at the corners to reflect light and images from these regions, allowing the image capture devices to obtain a comprehensive view without requiring the patient to reposition their feet. Additionally, convex mirrors 125 are integrated to reflect images from different angles, providing a broader field of view and capturing areas that might otherwise be obscured. The use of both concave and convex mirrors ensures that all relevant areas of the feet are thoroughly inspected, highlighting the innovative aspects of the present disclosure for comprehensive and efficient inspection.

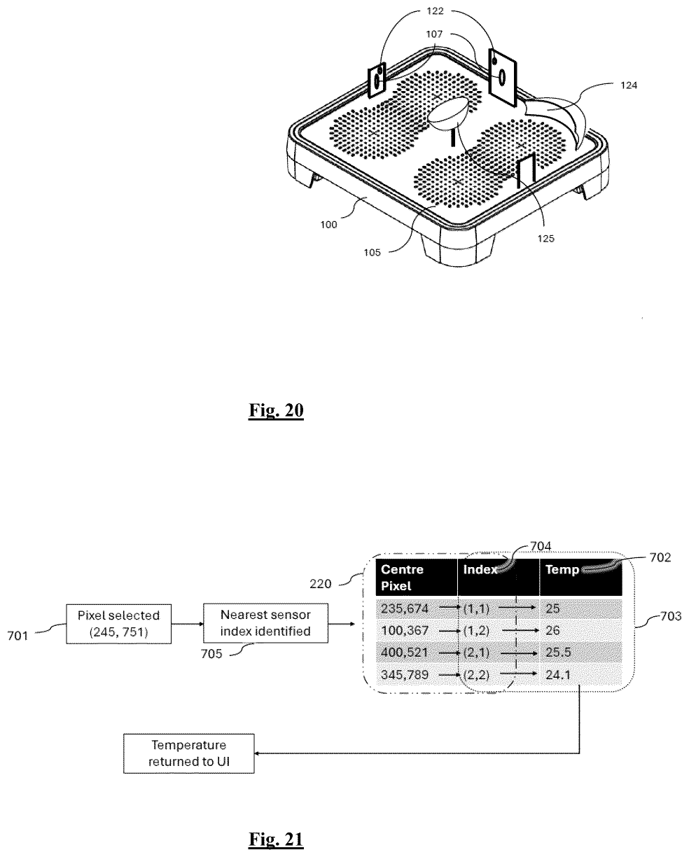

FIG. 21 illustrates a sensor indexing example within the context of a skin inspection system, showcasing the process of correlating a selected pixel in the visual data with the nearest temperature sensor to retrieve the relevant temperature data. The process is facilitated by a map 220, which is a data structure linking pixel coordinates to sensor data. This map is organized into columns, including “Centre Pixel,” “Index,” and “Temp.” The column headers for the Index 704 and Temperature 702 are explicitly labeled, and the data values within the temperature column are collectively referenced as 703. This structure enables the simultaneous evaluation of visual and thermal data by ensuring that temperature readings can be accurately correlated with specific locations on the visual image.

The process flow begins with the initial selection of a pixel 701 in the visual inspection pane, for example, at coordinates (245, 751). This selection is typically made by a user clicking on a point of interest. Following this, the system proceeds to the next step where the nearest sensor index is identified 705. This is achieved by using the map 220 to calculate the distance between the selected pixel and the “Centre Pixel” coordinate of each sensor.

Based on the shortest distance, the nearest sensor is identified. In this example, the nearest sensor is found to be the one with index (1,1). The corresponding temperature value from this sensor's data 703 is then retrieved from the map and returned to the user interface (UI), allowing the user to efficiently correlate the visual data with the thermal data.

This detailed description of FIG. 21 provides an in-depth understanding of the components and their function within the sensor indexing process, highlighting the innovative aspects of the present disclosure for efficiently correlating visual and thermal data during skin inspections.

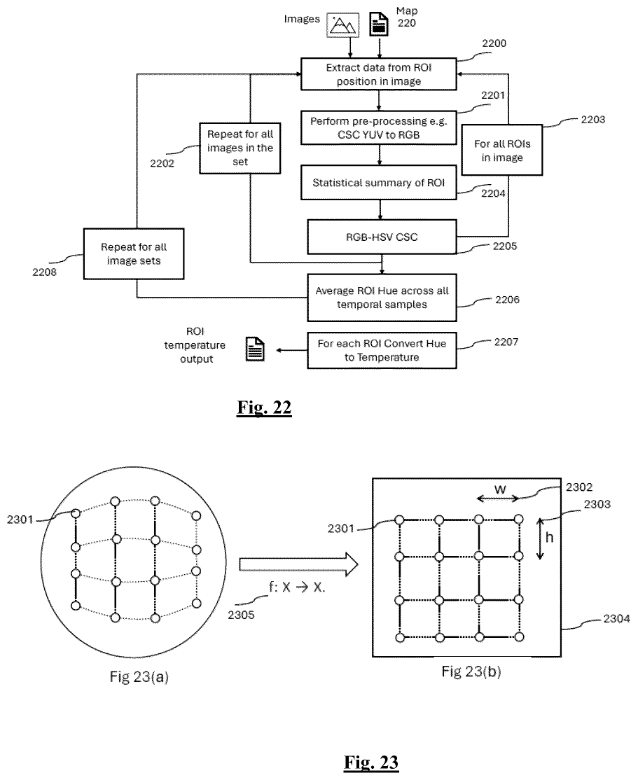

FIG. 22 illustrates a flow chart detailing the process of extracting and analyzing Region of Interest (ROI) temperature data from images captured by a skin inspection device. This process converts the color information in the regions of interest into temperature readings, enabling simultaneous evaluation of visual and thermal data. The key steps in this process are described as follows.

The process utilizes the map 220, a data structure that links pixel coordinates in the visual inspection pane to their corresponding sensor indices and temperature values. This facilitates the extraction of ROI data from the images based on predefined positions.

The process begins as the system proceeds to extract data from the ROI Position in image, step 2200. Using the map 220, this step identifies and isolates the relevant data from the specified ROI coordinates in each image. The extracted ROI data then undergoes pre-processing.

Next, the system will perform pre-processing, step 2201, which may include transformations like Color Space Conversion (CSC) from YUV to RGB. This ensures the data is in the optimal format for subsequent analysis. The data extraction and pre-processing steps are performed individually for each region of interest, as indicated by the nested loops to repeat for all images in the set, step 2202, and for all ROIs in the image, step 2203. This ensures accurate data extraction across all ROIs in every image within a given set.

Once the data is pre-processed for each ROI, a statistical summary of the ROI, step 2204 is generated. This summary may include metrics such as the mean, median, mode, and standard deviation of the color values within the ROI, helping to characterize the ROI data.

Following this, an RGB-to-HSV Color Space Conversion (CSC), step 2205, is performed. This step converts the color data to the HSV (Hue, Saturation, Value) color space, which is often used for color analysis because it separates color information (hue) from intensity (value), making it easier to analyze.

To improve accuracy and reduce noise, the system then calculates the average ROI Hue across all temporal samples, step 2206. This step 2206 involves averaging the hue values of the ROI across all images captured over time to obtain a more stable and accurate representation of the temperature.

The final conversion step is to Convert Hue to Temperature for each ROI, step 2207. This step 2207 converts the averaged hue values into temperature readings based on a predefined calibration, resulting in a temperature output that corresponds to the visual data.

Finally, the entire process is repeated for all image sets, step 2208. This ensures that a comprehensive analysis is performed across multiple datasets, which may represent different time periods, conditions, or patients.

This detailed description of FIG. 22 provides an in-depth understanding of the components, their functions, and interconnections within the process of extracting and analyzing ROI temperature data, highlighting the innovative aspects of the present disclosure for efficiently converting visual data to thermal readings in a skin inspection system.

FIG. 23 illustrates the process of de-warping an image captured using a fisheye lens to correct the geometric distortions and map the temperature sensor array to a rectilinear grid. This process is essential for accurately correlating the visual and thermal data captured by the skin inspection device. FIG. 23(a) shows the initial image captured by the fisheye lens, which contains a visible rectilinear grid 2301. Due to the wide-angle properties of the lens, this grid appears distorted, with its lines appearing curved. This warped grid is used as a reference for the correction process.

A de-warping algorithm or transformation 2305, represented by the function f: X->X, is applied to correct these geometric distortions. The result of this transformation is shown in FIG. 23(b), which displays the de-warped image 2304. In this corrected, rectilinear format, the grid 2301 now consists of straight horizontal grid lines 2302 and vertical grid lines 2303. This corrected image accurately represents the original scene and allows for the precise mapping of temperature sensors to the visual data.

The overall process, therefore, involves capturing the initial warped image as in FIG. 23(a), identifying the reference points on the curved grid lines, applying the de-warping algorithm 2305 to map the curved lines back to a rectilinear format, and generating the final, corrected de-warped image 2304 as in FIG. 23(b).

The de-warping process can be summarized in the following steps. First, the initial image is captured using a fisheye lens, resulting in a warped image with both horizontal and vertical grid lines appearing curved. Next, the horizontal 2302 and vertical 2303 grid lines in the warped image are identified to serve as reference points for the de-warping process.

A de-warping algorithm is then applied to the image. This algorithm uses the known geometry of the rectilinear grid to correct the distortions caused by the fisheye lens, effectively mapping the curved lines back to straight lines and restoring the original rectilinear format. Finally, the process generates the de-warped image 2304. This corrected image accurately represents the original scene with straight grid lines and removed geometric distortions, which allows for the precise mapping of the temperature sensors.

Step 1: Capture Warped Image: The initial image is captured using a fisheye lens, resulting in a warped image with both horizontal and vertical grid lines appearing curved.

Step 2: Identify Grid Lines: The horizontal 2302 and vertical 2303 grid lines in the warped image are identified. These lines serve as reference points for the de-warping process.

Step 3: Apply De-warping Algorithm: A de-warping algorithm is applied to the image. This algorithm uses the known geometry of the rectilinear grid to correct the distortions caused by the fisheye lens. The algorithm maps the curved lines back to straight lines, restoring the original rectilinear format.

Step 4: Generate De-warped Image 2304: The result of the de-warping process is a corrected image where the grid lines are straight, and the geometric distortions have been removed. This de-warped image accurately represents the original scene and allows for precise mapping of the temperature sensors.

This detailed description of FIG. 23 provides an in-depth understanding of the components, their functions, and interconnections within the de-warping process, highlighting the innovative aspects of the present disclosure for correcting geometric distortions in images captured by a fisheye lens in a skin inspection system.

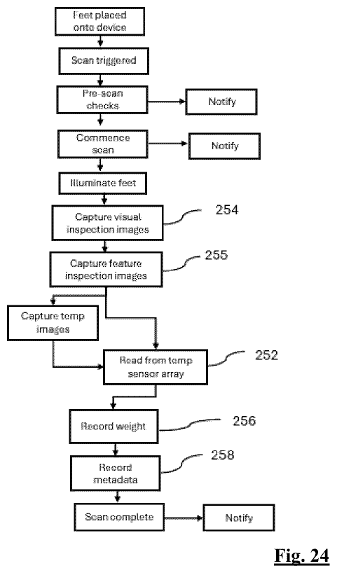

FIG. 24 illustrates a flowchart detailing the scan capture sequence of a skin inspection device designed to identify abnormalities, such as diabetic foot ulcers. The flowchart outlines the step-by-step process from the initial placement of the feet onto the device to the completion of the scan, including the capture of visual, feature, and temperature data. The process begins when the patient's feet are placed onto the device, which then causes the scan to be triggered automatically. Following this, a series of pre-scan checks are performed to ensure conditions are suitable for an accurate scan, such as verifying foot placement and checking for obstructions, and ensuring the user is not moving. If any issues are detected during these checks, a Notification is sent to alert the user or operator to address the problem.

Once the pre-scan checks are successfully completed, the system will commence the scan, and may continue to execture some or all of the prescan checks on the images as they are captured. Another notification may be sent to inform the user that the scan is in progress. The device then proceeds to illuminate the feet, ensuring the captured images are clear and well-lit for accurate inspection.

The core data acquisition phase involves capturing multiple types of data. The system proceeds to capture visual inspection images, step 254, to provide a detailed view of the skin's surface for identifying visible abnormalities. This is followed by the capture of feature inspection images, step 255, which involves capturing additional images with settings optimized to highlight specific features like texture or color variations. Concurrently or sequentially, the system will capture temp images using the embedded temperature sensors. The system may process these images to perform feature detection (e.g. bandage detection by analysing a hue histogram of the foreground object), image correction e.g. cropping and stitching images to reduce image size, image enhancement or image quality checks (e.g. checking that images are not corrupted by checking specific image data against expected known values within the scene), The data from these sensors is then processed by the read from temp sensor array, step 252, which provides quantitative measurements of the skin's temperature at various points.

Following the image and temperature data acquisition, the system will record the patient's weight, step 256, to provide additional context for the scan. Finally, the device will record metadata, step 258, which may include the date and time of the scan, ambient conditions, and device status.

Once all necessary data has been captured and recorded, the scan is complete. A final notification is then sent to inform the user or operator that the scan has finished and the data is ready for review and analysis.

This detailed description of FIG. 24 provides an in-depth understanding of the components, their functions, and interconnections within the scan capture sequence, highlighting the innovative aspects of the present disclosure for efficiently capturing and recording both visual and thermal data during skin inspections.

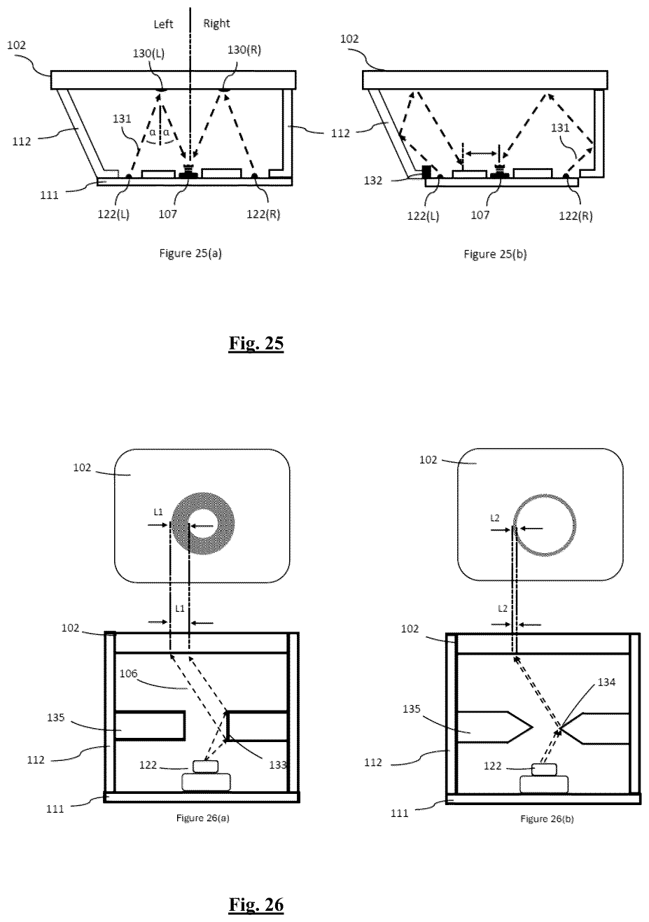

FIG. 25 illustrates cross-sectional views of a skin inspection device designed to identify and mitigate the effects of direct and indirect light reflections, which can affect the accuracy and quality of the captured images. The figures demonstrate how different geometric configurations of the device can reduce glare and unwanted reflections.

FIG. 25(a) shows a configuration where direct illumination can cause unwanted reflections. The top surface of the device is the transparent panel 102, where the patient places their foot for inspection. The entire assembly is supported by a base 111 and enclosed by sidewalls 112. Illumination is provided by a left light source 122(L) and a right light Source 122(R) to ensure the area is well-lit. Positioned centrally within the hollow interior is the image capture device 107, typically a camera, which captures images of the foot.

In this setup, the dashed lines representing the light rays 131 show how light from the sources can reflect off the underside of the transparent panel. This creates direct reflections at the left reflection point 130(L) and right reflection point 130(R), which can cause glare in the captured image and negatively impact image quality.

FIG. 25(b) demonstrates an improved configuration designed to reduce glare. While it includes the same core components like the transparent panel 102, image capture Device 107, and light sources 122(L), 122(R)), key modifications have been made. The sidewalls 112 in this configuration are angled to help manage and reduce indirect reflections. More importantly, a baffle 132, which is an angled structure, is positioned within the device. The baffle is specifically designed to manage the path of the light rays 131 by directing them away from the image capture device. This configuration helps to minimize the impact of indirect reflections on image quality.

In summary, FIG. 25(a) demonstrates how direct illumination can create glare at reflection points 130(L) and 130(R), while FIG. 25(b) shows how the use of angled sidewalls and a baffle 132 can effectively reduce these reflections, leading to improved image quality.

FIG. 26 illustrates how the geometry of a light source aperture can impact image artifacts by demonstrating two configurations and their effects on light reflections. Both configurations are shown within a skin inspection device that includes a transparent panel 102 on top, a base 111 for structural support, and sidewalls 112. A cover 135 protects the internal components, including a light source 122 which provides illumination for the foot.

FIG. 26(a) demonstrates a configuration with a vertical wall aperture 133, which is an opening with vertical walls positioned around the light source. The light ray path (L1) shows how light emitted from the source can reflect off these vertical walls and then off the underside of the transparent panel. These indirect reflections can cause unwanted glare and image artifacts, thereby reducing image quality.

FIG. 26(b) shows an improved configuration featuring a knife edge aperture 134. This is an opening with a sharp-edged design that minimizes the height of the vertical walls, reducing the likelihood of indirect reflections. The corresponding Light Ray Path (L2) demonstrates that light is directed away from the transparent panel 102 due to this sharp-edged design. This configuration significantly reduces indirect reflections and improves image quality by minimizing glare.

The knife edge aperture 134 is designed to manage light and reduce image artifacts. In this context, a “knife edge” refers to the very sharp, thin terminating edge of a physical component, which acts as a precise boundary for light to minimise unwanted scattering and reflection.

As shown in the cross-section, the aperture is formed by the bevelled surfaces of the component 135 converging to create a pointed, V-shaped profile aimed directly towards the light source 122. This design is a key distinction from a simple vertical-walled opening, as it is specifically engineered to minimize the vertical surface area that could otherwise cause reflections.

The function of this physical configuration is to act as a superior light baffle. The sharp point of the knife edge 134 intercepts and blocks stray light rays emitted at high angles from the source 122. By eliminating the reflective vertical surface found in alternative designs (as in FIG. 26a), the knife edge ensures that any incident stray light is absorbed or reflected away from the camera's field of view. The result, as illustrated by the clean light ray path (L2), is that only a controlled cone of light passes directly to the inspection area on the transparent panel 102. This prevents contaminating internal reflections, significantly reducing glare and improving the quality of the captured image.

The significance of the knife edge aperture design is visually demonstrated in FIG. 26 by comparing the width of the resulting light artifact, L2, with the artifact L1 produced by a standard vertical wall aperture. The wider artifact L1 represents a prominent ring of stray light reflected from the vertical wall, creating significant glare that can oversaturate the image sensor and render the area it covers effectively unusable for inspection.

In contrast, the substantially narrower width of L2 shows that the knife edge aperture 134 has successfully suppressed these reflections. By minimizing this artifact, the knife edge design significantly reduces the ‘dead zone’ on the inspection surface, thereby increasing the usable area available for valid data capture and enabling a more comprehensive analysis.

Furthermore, this improvement in light control also enhances data integrity. The intense light from a wide artifact like L1 can bleed into adjacent pixels and corrupt data from nearby sensors. The smaller, more contained artifact L2 minimizes this effect, ensuring that the data captured remains more accurate and reliable.

Ultimately, the reduced width of L2 is not merely an aesthetic improvement; it is the visual evidence that the knife edge aperture's physical configuration provides a functionally superior solution to the technical problem of internal reflections. This leads directly to a cleaner image, a larger effective inspection area, and more trustworthy data.

In summary, this detailed description of FIG. 26 highlights the innovative aspects of using a knife edge aperture to manage light reflections, thereby enhancing image quality during skin inspections.

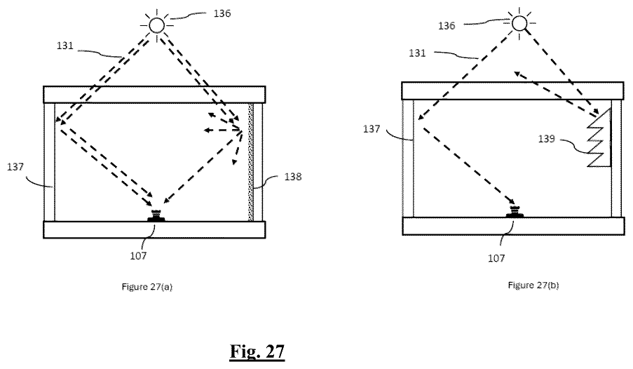

FIG. 27 illustrates how different surface finishes and geometries can impact ambient light noise within a skin inspection device. The focus is on how these design elements can reduce unwanted reflections and improve image quality by managing the paths of light rays 131 that originate from an external ambient light source 136, such as sunlight or room lighting. These light rays interact with the internal surfaces of the device before reaching the centrally located image capture device 107.

FIG. 27(a) demonstrates the difference between two types of surface finishes on the sidewalls. A smooth surface finish 137 tends to reflect light in a more focused and direct manner. As shown, the light rays 131 striking this smooth surface are reflected directly into the image capture device, potentially causing glare and reducing image quality. In contrast, a textured surface finish 138 diffuses the reflected light, scattering it in multiple directions. This disperses the light rays 131, reducing the amount of light that reaches the image capture device and thereby minimizing glare and improving image quality.

FIG. 27(b) illustrates the impact of sidewall geometry on managing ambient light reflections. A vertical sidewall 137, which extends straight up from the base, can cause reflected light rays 131 to be directed into the image capture device, resulting in glare. The improved design features an angled sidewall with optical baffles 139. These baffles are structures specifically designed to absorb or redirect light, preventing it from reaching the image capture device. As shown, the light rays 131 striking this angled surface are deflected away from the image capture device, which reduces glare and improves image quality.

This detailed description of FIG. 27 provides an in-depth understanding of how these design elements function to manage ambient light reflections, highlighting the innovative aspects of the present disclosure for improving image quality during skin inspections.

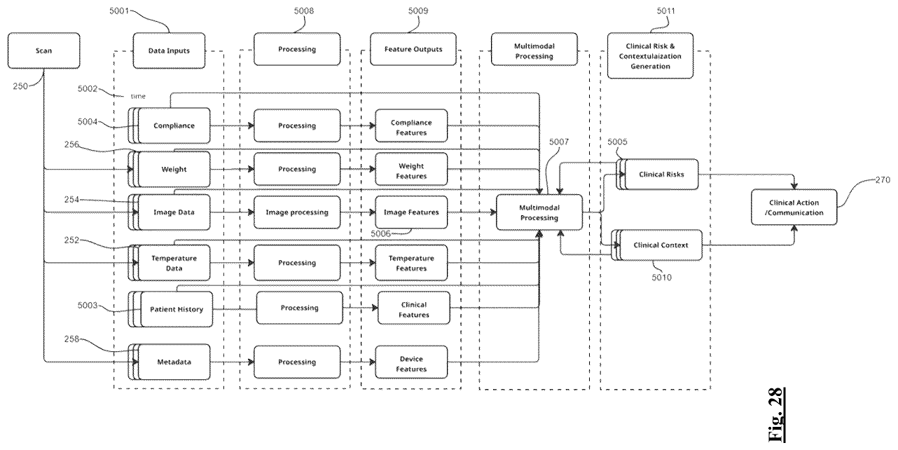

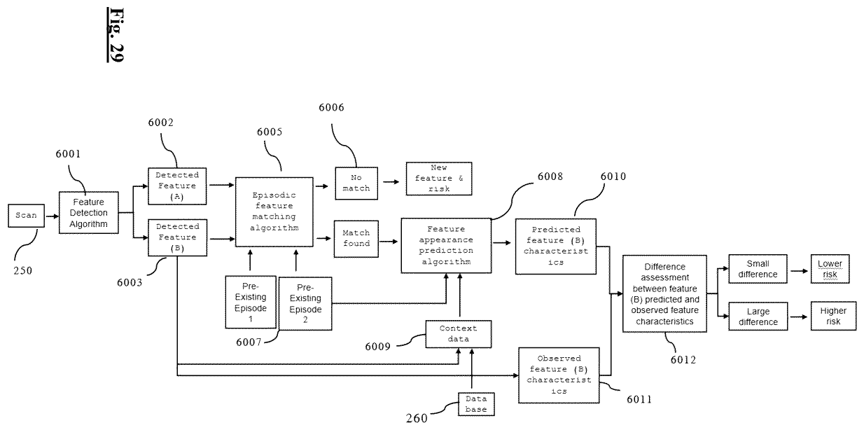

FIG. 28 illustrates a system diagram for an automated scan inspection monitoring system incorporating multi-modal processing 5007. The diagram illustrated how a variety of scan 250 and other data inputs 5001 are processed 5008 to generate new data features 5009 that may be used in alone or in combination with raw data inputs 5001, historical time series data 5002 to generate clinical risks 5005 and context 5010 that can drive clinical communication 270.

The element 5002 is used to indicate historical time series data as stacked icons and is visible in data inputs, 5001, clinical risk and contextualization 5005, 5010, and though not shown in the diagram for clarity, historical data may also be present and used for feature outputs 5009 and clinical actions/communication 270.

### System Operation and Alternative Embodiments

The skin inspection device 100 is a highly configurable system. The following sections describe its operational details, technical background, and various alternative embodiments.

### Processor Control and Image Sensor Configuration

As shown in the image capture arrangement of FIG. 5, the system includes an image capture device 107 with a lens 121 and multiple illumination sources 122, all controlled by one or more illumination drivers and a programmable processor. The image sensor within the device 107 allows for numerous parameters to be adjusted, including image resolution, binning, color space, contrast, brightness, gamma, auto-white balancing gains, frame rate, compression, and various correction and cancellation settings (e.g., lens correction, defect pixel cancelling, noise cancelling).

The programmable processor is responsible for programming these image sensor parameters, as well as reading, storing, processing, and transmitting images. It also controls and monitors the scene illumination by setting the rate and intensity of the illumination driver, which can be controlled via Pulse Width Modulation (PWM), current, or voltage signals. The processor can turn the driver on or off, implement a soft start for smooth power-on, and monitor the driver for faults such as undervoltage, overvoltage, or open/short circuit conditions. Example illumination drivers may include LP8861 drivers. Furthermore, the processor can program individual control of LEDs or groups of LEDs to provide additional illumination in specific regions of the image where vignetting may be more prevalent.

### Illumination Sources and Stability

The illumination sources 122 may comprise LEDs, CFL tubes, or filament bulbs. The color temperature and color rendering index (CRI) of the sources are selected to maximize sensitivity for the measured parameter. For example, a neutral white color temperature with a high CRI is advantageous for measuring thermochromic hues. Illumination sources with high and stable intensity are preferred to minimize exposure time and the effects of ambient light. The illumination driver must ensure the stability of the light source during image capture. Any failure of the light source can be detected by current sensors or changes in voltage, allowing the processor to take remedial action, such as flagging an error or re-attempting the capture.

### Multiple Cameras and User Display:

In a preferred embodiment, the skin inspection device 100 contains four image capture devices 107, with two positioned underneath each foot for comprehensive coverage. The device may also have an LCD screen to communicate information to the user, such as scan results, progress reminders, and information about foot placement. This screen can be integrated, mounted on a pole, or installed on a wall. It can connect wirelessly via Bluetooth or other protocols and may be implemented as an app on a user's phone, tablet, or computer. The display can also provide audio feedback.

### Weight Measurement and Scan Triggering:

In preferred embodiments, the device is operable to record the user's weight using conventional load cells. A scan can be triggered automatically by the detection of a weight change when a user steps on the device 100. Alternative triggering methods include a “tap-to-wake” interaction, proximity sensors (ultrasound or infrared), voice commands, or buttons within a software application. The device 100 can also be configured for seated use with a lower weight threshold, which is useful for patients at risk of falls.

### User Identification and Access Control:

The device 100 can be configured to identify a user based on various characteristics, including foot size, shape, color, texture, temperature, or weight. This identification allows the system to link the scan to the correct patient's profile in the data monitoring system 259. This feature can also be used to control data access; for example, in a household, the device might send a full scan for review for an at-risk user but function only as a standard smart scale for other users.

### Pre-Scan Checks and Positional Guidance:

The device 100 may perform various pre-scan checks, such as a soiling inspection for dirt or debris on the transparent panel 102, checks for incorrect foot placement or foreign objects like socks, or checks for a stable weight reading to ensure the patient is not moving. If an issue is detected, the user is notified to correct it. To assist with proper positioning, the device can provide audible feedback or visual cues on a screen. For example, lights on the device could guide the user to adjust their foot position. A glow-in-the-dark foot silhouette may also be provided on the surface to aid placement in low-light conditions.

### Imaging Non-Plantar Surfaces and Advanced Imaging:

As shown in FIG. 20, the device can include additional image capture devices 107 and illumination sources 122, or use concave 124 and convex 125 mirrors, to capture images of non-plantar surfaces like the top and sides of the feet. In systems with multiple cameras, an area of overlap is advantageous for stitching images together to create a 2D or 3D visualization of the whole foot. In another embodiment, the light sources 122 and image capture device(s) 107 could be configured to operate as a pulse oximeter to monitor blood oxygen saturation levels. In a further embodiment the image sensors may comprise hyperspectral image sensors use to monitor tissue oxygenation state.

### Digital Imaging and Image Sensors

This skin inspection device utilizes digital photography, where an image sensor 904 containing a dense array of light-sensitive photodetectors replaces traditional photographic film. Light reflected from the target is focused by a lens 121 onto the image sensor 904 to form an image. The process of digitizing the image facilitates its digital processing, storing, transmitting, and viewing, and allows for the automated or manual extraction of information such as the shape, size, color (hue), and brightness of objects.

### Sensor Technology and Color Capture

The core of the system is the image sensor 904, which can be fabricated using various processes, such as CMOS (resulting in CMOS Image Sensors or CIS) or CCD (Charge-Coupled Device). To capture color information, a color filter array (CFA) 903 is typically placed directly on the sensor surface during manufacturing. This filter ensures that each photodetector corresponds to a specific color (e.g., red, green, or blue). In a conventional image sensor three color channels are present as an output. However Hyperspectral The voltage signal from each photodetector, which varies with light intensity, is then digitized by an analog-to-digital converter (ADC) and stored in memory. The number of photodetectors on the sensor directly corresponds to the number of pixels in the final image.

### Image Formation and Processing

The format of the image sensor 904, in conjunction with the focal length of the lens 121, determines the camera's Field of View (FOV). A lens mount 909 provides mechanical stability for the optical and electronic components necessary to accurately record the object. The array of signals from the image sensor 904 is constructed into a digital image, which may be stored in a RAW format or converted to other formats like JPEG, HEIF, or TIFF.

Signal processing, including white balance adjustment, sharpening, and noise reduction, can occur directly on the image sensor 904 or in a camera module that incorporates a Printed Circuit Board (PCB) 905. This PCB can house additional electronics, such as Integrated Circuits (IC) 906, Input/Output (I/O) connectors 907, and specific signal processing electronics 908 to process the output from the sensor.

### Impact of Sensor Size

Sensor size is a critical factor that directly impacts image quality and sensitivity. Larger sensors offer a greater dynamic range and lower noise levels, which helps in revealing details in both bright and dark areas of the image and improves performance in low-light conditions. Sensor sizes range from large Full-Frame (36 mm×24 mm) sensors used in high-end systems to smaller formats like APS-C, Micro Four Thirds, 1-inch, and 1/2.3-inch sensors used in more compact and portable devices.

### Optics and Lenses

In a camera, the function of the lens 121 is to focus incoming light onto the image sensor 904, where it can be converted into an electrical signal. The lens 121 may be a single lens or a compound lens system, and can be made from glass or plastic with refractive or diffractive properties. To minimize device thickness, a thin focusing optic like a Fresnel lens could be used. The lens is designed to converge light rays to a focal point on the image sensor 904 to produce a sharp image, and may have an adjustable or fixed focal point.

A wide-angle lens 121 is used to produce a large Field of View (FOV), which is necessary when the object and image capture device are in close proximity. The FOV, typically expressed in degrees, refers to the extent of the observable scene captured by the imaging system. While wide-angle lenses have a reduced depth of field, this is not a problem in this application where the object is flat and at a fixed distance.

### Optical Aberrations and Corrections:

Imperfections in the image produced by the lens 121 are known as aberrations. Spherical aberration, which causes blur at the edge of the image, and chromatic aberration, which causes color distortion, can be corrected by adding additional lenses to the system. Chromatic aberration is a particular concern in applications where hue is measured, as it occurs when a lens fails to focus all colors of light to the same point. The Modulation Transfer Function (MTF) of a lens measures its ability to accurately reproduce detail, quantifying contrast and sharpness.

### Aperture, Depth of Field, and Field of View:

The camera may have an aperture to control the amount of light reaching the image sensor 904 and to define the depth of field. In an application where the object distance and illumination are fixed, the aperture can be locked to a suitable value. The field of view can also be locked if the object size and distance are fixed, or a plurality of cameras can be used to stitch together a combined field of view that covers the entire object.

### Image Sensor Configuration

Various configuration parameters of the image sensor can be adjusted to optimize the appearance of the captured image.
Auto White Balance (AWB): This is the adjustment of the gain of the red and blue channels to achieve accurate color in different illumination levels.
Dynamic Range: This is the ratio between the brightest and darkest parts of an image that a sensor can capture. A higher dynamic range allows for more detail to be recorded in both highlights and shadows.
Exposure: This refers to the amount of light that reaches the sensor and is controlled by the aperture, shutter speed, and ISO settings.
Gamma Correction: This is the process of adjusting the brightness and contrast of an image to correct for the non-linear way humans perceive light and color.
Resolution: This is the amount of detail a camera can capture, measured in pixels. Higher resolution sensors collect greater detail. Common image formats based on the number of photodetectors include 1280×720, 1600×1200, and 2592×1944 (often referred to as 5-megapixel).
Sampling (Binning): This technique combines data from multiple adjacent pixels into a single “super pixel” to improve performance in low-light conditions by reducing noise and increasing sensitivity.
Bracketing: This technique, also known as exposure stacking, involves taking multiple images at different settings for illumination, ISO, or shutter speed to ensure no details are lost and to increase the dynamic range.
Pixel Patterns: The distribution of color filters on the sensor pixels can follow various patterns. A typical Color Filter Array (CFA) 903 is the Bayer filter array, where two out of every four pixels are green (G), one is red (R), and one is blue (B), reflecting the human eye's stronger sensitivity to green light.
Spectrum: the visual wavelength we

### Illumination

The illumination sources 122 may include LEDs, CFL tubes, or incandescent bulbs. For applications where color (hue) is measured, an ideal source would have a flat spectral power distribution. Since no artificial source is perfect, a source with a high Color Rendering Index (CRI) is desirable. The perceived color of the light source is described by its Correlated Color Temperature (CCT).

The intensity (brightness) of LEDs can be controlled by changing the supplied current or by using a neutral density (ND) filter 902. The light from LEDs is randomly polarized, but it can be advantageous to polarize it using a linear polarizing filter 902 to reduce glare. It is important to allow LEDs to settle before an image is captured, as their light intensity and spectral power distribution change over time.

### Object Properties: Human Skin

The formation of an image is dependent on light reflected from the object being captured in the camera lens 121. In this disclosure, the object of interest is the human skin. Imaging the skin is a complex process due to the interaction of light with its surface and subsurface structures. Skin is translucent; while some light reflects from the surface, most (>90%) transmits into the skin, where it undergoes absorption, reflection, and scattering. Light absorption is dependent on the composition and density of hemoglobin (found 50-500 μm below the surface) and melanin (in the top 50-100 μm of the epidermis).

Subsurface scattering occurs between skin layers. Rayleigh scattering occurs from subcellular structures, while Mie scattering occurs from larger structures like collagen and melanosomes. Light that undergoes Rayleigh scattering becomes increasingly polarized as the scattering angle approaches 90°, an effect that can be leveraged by using a linear polarizer to form an image that selectively includes this light. Furthermore, as pressure is applied to the skin, the reduced blood flow leads to blanching (whitening). The time it takes for color to return after pressure is removed can be used to identify issues with blood flow. This skin blanching information can be captured through a series of images taken at different timepoints.

### Glare and Mitigation Techniques

Glare occurs when the RGB channels on an image sensor become saturated (e.g., reaching a value of 255 in an 8-bit system, resulting in a white pixel). It can be caused by specular reflections from a transparent surface, such as when the panel 102 acts as a mirror, or by a refractive index mismatch between two mediums, such as the glass panel and air, which can lead to internal reflections.

Glare can be reduced or eliminated through several methods. Anti-reflective coatings (ARC) 901 or films can be used to alter the refractive index of the panel 102, allowing more light to pass through. An ARC can be created by placing a specific filter 902 in front of a lens or by fabricating it directly onto the lens 121. Using a polarizing filter (which can be used in conjunction with an Infrared filter (902)) or diffusers can also reduce glare by polarizing the light or increasing its uniformity. Additionally, glare can be managed by appropriately configuring the light source and sensor settings for the specific image scene.

### Color Space and Post-Processing

Image sensors can output images in various formats (e.g., RAW RGB, RGB565, YUV422, YCbCr422) that represent different ways of coding color and brightness information. While formats with increased color and spatial information can reduce quantization error, they also increase file size, which is an important consideration for data transfer over cellular networks.

Different color spaces can be used for analysis. YUV is an alternate color space that balances accuracy and file size by representing colors with one luminance (brightness) component and two chrominance (color) components. HSV (Hue, Saturation, Value) is particularly advantageous for color measurements as it defines color as a single quantity (hue) in a polar coordinate, reducing computational requirements. The LAB color space may also be useful for detecting small changes in color differences. A greyscale image, which is composed exclusively of shades of gray, can be useful for highlighting visual features and has a smaller file size.

The outputted digital image may undergo post-processing to enhance features for inspection. Color correction, including white balance and gamma correction, can be used to adjust the representation of colors. Compression is used to reduce the file size of an image. Lossless compression (e.g., PNG, TIFF) reduces file size without losing any image data, while lossy compression (e.g., JPEG) significantly reduces file size by discarding some data, which results in a reduction in image quality.

### Noise and Minimizing Variation

Signal noise in digital images can be caused by inconsistent illumination from ambient light sources (e.g., sunlight, room lights) or by inconsistencies in the light intensity measurements made by the image sensor 904. The impact of noise can be reduced by minimizing variation in the illumination of the object and in the acquisition conditions of the imaging system.

### Minimizing Undesired Illumination and Variation

Illumination variation can be minimized by ensuring consistent settings for the illumination source(s) between scans and by minimizing the impact of ambient light conditions. In general, desired light from the primary illumination sources 122 (e.g., LEDs) reflected from the target should be maximized, while undesired light reflected from other surfaces-which can cause stray reflections, image glare, and other artifacts-should be minimized. Light from secondary sources like room lights or daylight, which creates ambient light noise, should also be minimized. Advantageously, the intensity of the primary illumination sources 122 can be set to a level high enough that variations in ambient light have a negligible effect on the object's illumination.

Mechanical Design to Reduce Reflections (with reference to FIGS. 25, 26, and 27)

Undesired illumination from primary sources can manifest as glare, reflections, and shadows. The severity of these can be minimized through the mechanical design of the device.

As illustrated in FIG. 25, both direct and indirect reflections can negatively impact image quality. In FIG. 25(a), a light ray 131 from an illumination source 122 hits the underside of the transparent panel 102 and reflects directly into the camera lens, creating a reflection point 130 that can cause glare and saturate the image sensor. In an alternative embodiment, the illumination sources can be operated independently, allowing one side of the panel to be illuminated by the source on the opposite side, thereby eliminating direct reflection points. Indirect reflections, as shown in FIG. 25(b), can occur when a light ray 131 reflects off a vertical side wall 112 and then into the image capture device 107. This can be mitigated by using features like an angled baffle 132 to constrain the light path and prevent it from striking the image capture device.

As shown in FIG. 26, indirect reflections can also be caused by apertures in the cover 135 over an illumination source 122. FIG. 26(a) illustrates how an aperture with vertical walls 133 can create a circular reflection on the transparent panel 102. The size of this reflection can be significantly reduced by incorporating a knife edge 134 on the aperture, as shown in FIG. 26(b). This design ensures that light rays from the illumination source cannot strike the upper surface of the aperture, preventing the indirect reflection from being visible in the image.

FIG. 27 demonstrates how surface finishes and geometry can reduce the impact of ambient light sources 136. In FIG. 27(a), a smooth surface finish 137 on a vertical sidewall reflects light rays 131 directly into the image capture device 107. In contrast, a textured surface 138 disperses the light rays, significantly reducing the amount of light reflected into the device. In FIG. 27(b), the geometry of the sidewalls is modified. While a vertical side wall 137 can reflect ambient light into the device, angled surfaces with optical baffles 139 can be used to absorb or redirect the ambient light away from the image capture device. These features can be combined; for example, the device's base could have light baffles and be made from a dark material with a high VDI surface finish.

### Managing Glare and Refraction

The size of a glare patch is related to the acquisition parameters of the image sensor. For instance, increasing the exposure time can cause a larger area of pixels around a reflection point 130 to become oversaturated. Therefore, it is advantageous to use acquisition parameters that reduce the size of the glare patch for regions close to reflection points.

It is also important to consider the impact of refraction, which occurs when light passes from one medium to another (e.g., from the air into the glass of the transparent panel). This can result in two offset reflections of a single feature-one from the bottom surface of the panel and one from the top-due to the change in the angle of the light rays.

### The Advantage of Fixed Acquisition Conditions

While image sensors are typically configured to automatically adjust settings like ISO, shutter speed, and white balance to adapt to changing ambient conditions, this can result in significant variations in the recorded color and brightness of an object across different scenes.

For a foot inspection device that can provide consistent conditions—such as the level of illumination, distance to the object, and position of artifacts—it is advantageous to fix the acquisition conditions of the image sensor. By fixing parameters such as depth of focus, resolution, ISO, shutter speed, color space, contrast, and white balance gains, variations between images captured at different times are minimized.

This consistent configuration ensures that the appearance of features remains constant across images, which is crucial for monitoring changes over time. It enables the reliable tracking of clinical features, such as the color changes associated with blanching, rubor (redness), or post-inflammatory hyperpigmentation, with a degree of accuracy that would not be possible if the acquisition settings were variable.

### Optimizing for Noise Reduction and Illumination Inconsistency

In general, the more light measured by the image sensor, the lower the noise in the recorded data. While this can be achieved by using a longer shutter speed, doing so risks overexposure and blurring if the object moves.

Furthermore, inconsistency in the level of illumination across the field of view can impact the visibility of features. Areas with lower illumination may appear dark or underexposed, while areas with higher illumination, such as near a reflection, may be overexposed and result in glare. To address these challenges, it is advantageous to use a multi-exposure configuration. In this approach, the acquisition conditions are optimized for different regions within the scene, and the optimized portions of the image are then combined to create a single high dynamic range (HDR) image. This allows for an image with ideal conditions across the entire scene, which is not possible with a single set of acquisition conditions.

### Optimizing Conditions for Intended Purpose of Image

In addition to using consistent illumination and acquisition conditions between scans, it is also advantageous to optimize these conditions for the specific purpose of the image being captured. The system can be configured to capture several types of images, each with tailored settings.

For example, visual inspection images 254, intended for review by a person, can be captured with acquisition conditions optimized to provide life-like images that closely match the visual appearance of the skin to the human eye. In contrast, feature inspection images 255 can be captured with settings optimized to increase the visibility of particular skin features. This might involve using a high exposure to enhance dark or small features (even if it overexposes the rest of the scene) or capturing an image with higher contrast to highlight skin texture.

In systems using optical response sensors like thermochromic sensors, images can be captured specifically for measuring the color of the material, which is then converted to a temperature reading. For these images, the acquisition conditions are preferably optimized to minimize color signal noise, for instance, by using intense illumination without oversaturating the image sensor pixels. Noise can also be reduced by taking multiple images at each configuration, a process enabled by the fixed position of the feet relative to the device's optics.

### Scan Capture and Data Processing Sequence

As shown in the flowchart of FIG. 24, the scan capture process begins when a user places their feet on the device, meeting the trigger conditions. A series of pre-scan checks are performed, and if any check fails, a notification is sent to the user via an audible alert, LCD display, or other means. If all checks pass, the scan commences, and the light sources 122 are enabled to illuminate the feet.

The system then captures the various image types, such as the visual inspection images 254 and feature inspection images 255. For thermal data, the system captures temperature images to be analyzed or, in the case of an electronic sensor array, records the temperature data directly. The patient's weight is measured from the load cells, and relevant metadata (time, date, ambient temperature, etc.) is recorded. Once the scan is complete, a notification is sent to the user, and the collected data is prepared as a scan payload 250 for transmission to the data monitoring system 259, which may involve file compression and data encryption.

### Region of Interest (ROI) Definition and Analysis

As illustrated in FIG. 9, when an image of the sensor array 105 on the transparent panel 102 is captured by the image capture device 107, the position of each sensor can be defined for analysis. In the captured digital image, a Region of Interest (ROI) 200 is defined for each sensor, and its precise location is noted by its central pixel coordinate 206. The size of this ROI, typically a square bounding box, can be varied (e.g., 3×3, 5×5, 7×7 pixels), with larger ROI sizes helping to reduce color signal noise. FIG. 22 details an example of a thermochromic ROI signal analysis chain. The processor takes a set of images and a map 220 containing a list of ROIs as input. For each ROI, the system measures the data at the ROI position 2200 and performs image pre-processing 2201 such as color space conversion. This is repeated for all ROIs in the image 2203 and for all images in the set 2202. A statistical summary of the ROI is analyzed 2204 (e.g., calculating the mean/median of pixel values), and the color space is converted to the desired output format, such as RGB to HSV 2205. Each ROI's value is then averaged across all image samples in the set 2206, and the final result is stored in an output file 2207. This process is then repeated for all other image sets 2208. In the case of Hue-to-Temperature conversion, these hue analysis results are converted to temperature values for the UI.

### Fisheye De-Warping Techniques

Fisheye lenses capture a wide-angle view by projecting incidental light onto a curved surface, which results in a spherically distorted projection of the world, as shown in the example of FIG. 6. In a fisheye image, straight lines become progressively more curved toward the edges of the lens. The process of de-warping involves converting this distorted image into a “rectilinear” or normal view by mapping the curved image data back into a flat, undistorted representation using mathematical transformations. These transformations recalculate the pixel coordinates from the fisheye image to their new positions in the rectilinear image.

Common de-warping techniques include:
Polynomial Mapping: Using polynomial equations to transform pixel coordinates.
Spherical to Cartesian Conversion: Mapping the spherical coordinates of the fisheye lens to the Cartesian coordinates used in flat images.
Interpolation: Using techniques like bilinear or bicubic interpolation to fill in gaps after determining new pixel positions, creating a smooth, undistorted image.
Geometrical Mapping: Deriving a model based on the lens geometry to perform the translation.
Homography Mapping: Transforming points from one plane to another, which is particularly useful when imaging a flat plane like a sensor panel.
Field of View Mapping: Adjusting the camera's field of view to match a desired perspective projection.
Radial Distortion Models: Applying a model to correct for radial distortion and translate it to a rectilinear perspective.
Deep Learning Models: Utilizing trained neural networks to perform the de-warping transformation.

### Fisheye De-Warping Using a Grid Array

As shown in FIG. 23, a fisheye de-warping transformation can be generated when a visible rectilinear grid 2301 with known horizontal 2302 and vertical 2303 spacing is present in the field of view. An example of such a grid is the printed rectilinear sensor array 207 shown in FIG. 9.

By identifying the grid keypoints in the warped fisheye image (as seen in FIG. 23(a)), the system can map them to the known, rectilinear grid (as seen in FIG. 23(b)). This is achieved by using a transform 2305 to map the set of keypoints from the warped image to their known rectilinear coordinates, which generates a homography matrix or an equivalent transform. This transform 2305 can then be applied to any subsequent images captured through the wide-angle lens to produce rectilinear images.

A key advantage of having a known rectilinear grid visible in the image is that it eliminates the need for costly and time-consuming calibration during the manufacturing process.

### Challenges in Mapping Datasets

A key challenge lies in analyzing the data collected by the skin inspection device 100, which simultaneously captures data from image capture devices 107 and an array of temperature sensors 105. Although the data is coincident-meaning it is both collocated (from the same physical region) and concurrent (from the same time)—the two datasets have different formats and geometries. The image capture device 107 produces a warped digital image, while the rectilinear sensor array produces a table of discrete temperature values. This disparity traditionally requires the datasets to be inspected independently.

To enable simultaneous inspection and reduce review time, it is highly useful to provide a map that relates the two datasets. For example, if an abnormality is seen in the visual data, the map would allow for concurrent assessment of the temperature data at that exact location. Conversely, a temperature anomaly could be instantly cross-referenced with the visual data to check for a visible injury.

However, creating such a map presents significant technical challenges:
1. ROI Detection and Indexing: The sensors appear as small Regions of Interest (ROIs) within the camera's field of view. The position of each ROI must be automatically detected and correctly indexed to align the sensor information with the corresponding image data. In an application with tens or hundreds of sensors, manually performing this task is impractical and prohibitively expensive. Incorrect indexing would lead to a mismatch between the visual and sensor data, risking completely inaccurate readings.
2. Manufacturing Variations: In a manufacturing environment with thousands of devices, variations in lens characteristics, sensor placement, and camera positioning will cause the sensor pattern and their corresponding ROIs to shift within the field of view from one device to another. This means a fixed template cannot be used, and each device requires its own unique mapping.

Therefore, a robust, automated method for detecting and indexing the sensor positions within the warped digital image is necessary to create the map that links the two datasets for simultaneous inspection.

### Challenges in Mapping Datasets: Distortion and Variation in Sensor Positions

A primary challenge in analyzing the collected data is mapping the two different datasets—the warped visual image and the discrete temperature values—so they can be inspected simultaneously. The use of a wide-angle lens, while necessary for close-proximity imaging, introduces several effects that complicate this process.

One effect is geometric distortion, where the geometry of an object appears altered in the captured image. As illustrated by the example in FIG. 6, a rectilinear checkerboard 207 shown in FIG. 6(a) appears as a distorted image 208 with curved lines in FIG. 6(b). This compression effect increases with distance from the center and alters the spatial relationship between features. For example, as shown in FIG. 7, while the physical distances between sensors in the rectilinear sensor array 105 may be equal (d1=d2), these distances appear unequal (d1′≠d2′) in the digital image 210 captured through the lens.

Another effect is the variation in the position of the Field of View 213 within the digital image 210, as shown in FIG. 8. Due to minor manufacturing differences, the position of the active image area can shift relative to the surrounding deadspace 211, as shown by the difference in x and y dimensions between FIG. 8(a) and FIG. 8(b).

As a result of this distortion and positional variation, it is necessary to have a means of automatically detecting and indexing the positions of the temperature sensors 105 within each unique distorted image. To facilitate this automatic and optimal sensor indexing, the example process flows shown in FIGS. 10 and 11 are provided. These methods eliminate the need for manual indexing and create a map 220 that links the two datasets, allowing them to be inspected simultaneously.

### Sensor Registration Process

FIG. 10 illustrates the sensor registration process, a method for automatically detecting and indexing sensor locations in an image. This process begins with two inputs: a source image with known ROIs 601, which acts as a reference, and a target image with unknown ROIs 608, which is the image requiring analysis.

First, both images undergo pre-processing 602, which can include resizing, noise filtering, or contrast enhancement to optimize them for the subsequent steps. Following this, key-point detection 603 is performed on both images to find common, identifiable points using algorithms such as SIFT, ORB, or SURF. These key-points are then matched, for example with a brute-force matching algorithm, to establish correspondences between the two images.

Once the key-points are matched, a homography matrix or transform is created 604. This transform mathematically maps the coordinates from the source image to the target image. This matrix is then applied to the known source image ROIs 605 to generate the initial positions and indices of the corresponding ROIs in the target image. To refine these positions, further ROI positioning optimization is performed 606 using a method like template matching (as shown in FIG. 12b), which iteratively searches for a more precise fit. The resulting ROIs are then inspected for quality 607 by evaluating their characteristics, such as ensuring the hues of thermochromic sensors are within an acceptable range. If poor quality metrics are found, further optimization is carried out to adjust the ROI box position to improve the metrics.

Finally, after the ROIs are accurately positioned and optimized, a map 220 is produced. This map links the final sensor coordinates in the target image with an index, allowing the information from different datasets to be accurately correlated.

### Contour Detection Indexing

FIG. 11 illustrates a flowchart for an alternative sensor registration algorithm using contour detection indexing. This process provides a systematic method for identifying and indexing sensor Regions of Interest (ROIs) in a warped image. The process begins with a target image with unknown ROIs 612 as its input. First, the system will mask the target image 613 by covering all sensor locations with one color (e.g., black) and the rest of the image with another (e.g., white). This simplifies the identification of the sensors.

Using this masked image, a contour detection algorithm is used to detect ROIs contours 614, identifying the shapes and locations of all sensors within the image. This can be performed using various computer vision methods, such as edge, blob, or ridge detection. The detected contours then undergo Filtering and pre-processing 615, where they are filtered to ensure they meet predefined thresholds for size and location, and the image may be pre-processed to compensate for lens distortion.

Next, the system will index the ROIs 616 by iteratively checking and mapping the contours against a known indexing pattern, such as a grid. Using a seed or starting contour, the algorithm populates a list by comparing each contour's position relative to a known contour until all are labeled.

Following indexing, the system will analyse target ROIs quality 617 by inspecting their characteristics, such as ensuring hues are within an acceptable range for thermochromic sensors. Based on this analysis, the system will adjust ROIs to maximise quality 618, which may involve repositioning an ROI's bounding box to optimize the quality metrics.

Finally, a map 220 is produced as the output. This map links the final sensor coordinates in the target image with an index, which can be used to associate the sensor's information with other datasets.

Output of the Detection and Indexing Process (with reference to FIGS. 13 and 21)

The output of the sensor detection and registration processes (shown in FIGS. 10 and 11) is a map 220 that contains information for each sensor, allowing for the association of coordinate information from one sensor domain to another. As shown in the example of FIG. 21, this allows the pixel coordinates 701 selected in a UI to be associated with a corresponding temperature value 702 from a temperature array by identifying the nearest sensor via a common index 704.

FIG. 13 illustrates how this map 220 can be represented using standard data structures. The map links the image sensor data, which is derived from the image 620, with the physical sensor data structures.

The Image Sensor Data may comprise:
A unique Index 621 for the sensor's position.
A measured Value 622 from the ROI (e.g., mean hue).
The Coordinates 623 of the ROI's center relative to an origin point 635.
The Size 624 of the ROI.
The capture Time 625.
Other 626 parameters determined from the image (e.g., Max/Min Hue).
Metadata 627 about the image capture conditions.

The Physical Sensor Data may comprise:
A matching Index 621.
A direct Sensor Value 628 (e.g., temperature).
The Datapoint Position 629 within its data structure, which could be a 1D array 632, 2D array 633, or 3D array 634.
The capture Time 630.
Other (631) statistics, such as variability over a specific time window.
   System Overview and Data Flow (with reference to FIG. 1)

FIG. 1 illustrates a skin abnormality detection system, showing the flow of data from the initial capture by the skin inspection device 100 to the final communication of potential abnormalities to the care team 151.

The process begins when a patient user 150 stands on the skin inspection device (100). The device captures multiple data types, including temperature data 252 from an embedded sensor array and visual image data 254 from image capture devices. It may also record weight data 256 and other relevant metadata 258. A map 220 is created to link the thermal and visual data.

All of this information is packaged into scan data 250, which is then sent to a data monitoring system 259 and stored in a database 260. A computer 300, potentially using algorithms, or a person using a graphical user interface (GUI) 301 inspects the scan data for abnormalities.

If an abnormality is found, a communication module 270 generates an abnormality alert 272 and sends the associated abnormality data 274 to the care team 151. The care team comprises the patient user 150, a healthcare professional 152 (such as a doctor or nurse), a provider 154 (such as a clinic or hospital), and a payor 156 (such as an insurance company), who can then take appropriate action.

### Configuration of the Data Monitoring System

The data monitoring system 259 provides various functionalities to aid in the management of patients at risk of developing abnormalities. The monitoring of data may be performed by humans, algorithms, artificial intelligence, or a combination thereof. For example, the system can be configured so that all received scans are initially reviewed by monitoring algorithms, with those determined to have potential abnormalities being forwarded for human review.

The system includes functionality to manage individual patient profiles, allowing for the recording of contact information, health information, next of kin, and communication preferences. It also supports the generation of device orders and can link a specific device to a patient's profile, ensuring that scan data 250 received from that device is correctly associated. This scan data (250), which is received into the database 260, can be reviewed in a First-In-First-Out (FIFO) manner, sorted by the time contained in the metadata 258, to ensure timely inspection.

### User Interface (UI) Overview

A user interface (GUI) 301, accessible via a web browser or application, allows users to interact with the system. The UI may include standard elements such as windows, menus, icons, and buttons, and can be controlled by various means including a mouse, touchpad, touchscreen, or voice control.

The Scan Inspection Pane (with reference to FIG. 14)

As shown in FIG. 14, the scan inspection pane 309 is a key feature of the user interface 301 that allows a user to inspect the scan data (250), which includes visual data 254, temperature data 252, weight data 256, and metadata 258.

This pane is organized with a series of tabs 310 that provide access to different functionalities:
A History tab can display the results of previous inspections, such as the history table 339 shown in FIG. 14.
A Notes tab allows the user to add relevant notes about the scan or patient.
A Compliance tab shows data related to patient adherence to scanning protocols, which may be displayed graphically as a donut chart 338.
A Zoom tab provides an enlarged view of the visual images.
A Device Info tab displays technical information about the patient's skin inspection device.
A Contact Info tab provides patient and clinician contact details.
An Annotate tab enables the annotation of features seen in the images, including adjusting image parameters and tagging visible features like calluses or ulcers.

The main feature of the scan inspection pane 309 is the visual inspection pane 312. This pane displays the visual data 254, which may be combined from multiple image capture devices to create a stitched image 305. It can display de-warped stitched images 303 or warped stitched images 304 and includes information such as the scan timestamp.

Interacting with Thermal and Visual Data

The UI can be configured to use the map 220 to enable simultaneous evaluation of visual and temperature data. A pointer 302 can track across the visual inspection pane 312, and upon a click, the system can highlight the selected sensor 306 that corresponds to the noted position. The selected sensor's temperature value 307 is then populated into the temperature inspection table 305.

This temperature table 305 can be configured to perform a temperature asymmetry inspection by comparing temperatures at key locations on the feet (e.g., Hallux, Metatarsal heads, Midfoot, Heel). It can also be configured to automatically change the data entry point as a user selects sensors sequentially on both feet, streamlining the inspection process.

As an alternative to individual sensor readings, the UI can support the inspection of entire regions, generating statistical results such as mean, max, or min temperature for that area. This can be done by a user selecting a region or by a computer vision algorithm that automatically identifies regions, populates a table, and flags scans for review if a temperature asymmetry assessment exceeds a certain threshold. Alerts can be delivered via on-screen messages, icons, or text messages.

To address the challenge of selecting small sensors 205 with a pointer 302, the UI can be configured to automatically determine the nearest sensor to a clicked location, as described in FIG. 16. The system identifies the selected pixel 308, calculates the distance to the central pixels of adjacent sensors, and highlights the closest one as the selected sensor 306, populating its temperature 307 into the table 305.

### Additional UI Controls

The inspection pane 309 also provides radio buttons 314 for rapid logging of common issues and scan toggle buttons 324 to switch between different scans for comparison.

Temperature data can also be displayed as a visual heatmap overlaid on the visual images. The heatmap controls 320 allow a user to toggle the heatmap, adjust its transparency, select different color scales, and specify the max/min temperatures for the color gradient. The heatmap can be rendered as discrete pixels corresponding to the sensors or as an interpolated map for a smoother appearance.

### Alternative Embodiments and System Flexibility

The disclosed method for generating the map 220, which relates sensor data to data from the image capture device, is highly versatile and applicable to various other types of sensor datasets.

For instance, the temperature sensor array can be replaced with other sensor arrays designed to measure different physiological parameters of tissue, such as impedance, pressure, lactate, pH, electrocardiology (ECG), or electromyography (EMG). Likewise, the image capture device is interchangeable with alternative imaging sensors, including contact imaging sensors, hyperspectral sensors, infrared sensors, or multiband sensors. f the sensor array within the output data and generating the map 220 remains the same, allowing for interaction with the results in the manner that has been described. f the sensor array within the output data and generating the map 220 remains the same, allowing for interaction with the results in the manner that has been described.

Furthermore, the graphical user interface features included in the figures are intended to be exemplary and are interchangeable with various alternative features that provide similar or equivalent functionality. For example, a toggle switch could be replaced with two check boxes. Similarly, the relative positions and sizes of the various features, panes, and tables are provided by way of example and are not intended to limit the disclosure in any manner.

### Individual and Population Management

The system is capable of generating various metrics from the collected data to support both individual and population-level management of patients.

### Metric Generation and Analysis

Metrics can be generated from individual point-in-time measurements or as longitudinal measurements over specified timeframes (e.g., last 3 days, last week, last month). These metrics may include a range of statistics such as mean, median, mode, max, min, and standard deviation, as well as the rate of change of any metric. To improve signal quality, filtering techniques like generating a moving average can be applied, which is particularly useful for metrics like weight that can vary throughout the day. c Metrics can also be combined to create more advanced indicators, such as risk scores, classifications, or foot health ratings. In one embodiment, an abnormality risk metric could be generated by multiplying the current peak temperature asymmetry by the rate of compliance over the last 7 days.

### User Grouping and Cohort Analysis

The system allows for the calculation of metrics across different groupings of users. Groupings can be based on:
Location: Region, state, or city.
Clinical Affiliation: Healthcare provider, health insurer, hospital, or clinic.
Demographics: Age, ethnicity, or gender.
Health Status: Duration of diabetes, BMI, weight, or history of ulceration or amputation.
Monitoring Results: Compliance, temperature asymmetry, weight change, visual abnormalities, or poor foot positioning.

### Intervention Thresholds and Risk Scoring

Metrics can be monitored for signals or markers that indicate potential abnormalities. An intervention threshold 333 can be defined for any metric, which, when crossed, triggers specific actions or interventions.

An exemplary embodiment of this is shown in the population tabular metrics 319 in FIG. 17. In this example, an abnormality risk score is calculated for each patient by dividing the maximum foot temperature over the last 3 days by the percentage compliance over the last 7 days. The intervention thresholds for this score are set as Level 1 (0-29.99), Level 2 (30-49.99), and Level 3 (>50). The intervention type varies accordingly: Level 3 (“A&E”) implies an emergency visit, Level 2 (“Clinic”) warrants a podiatry visit, and Level 1 (“Contact”) requires notifying the patient of a potential issue. the patient is particularly effective, as neuropathy may prevent them from sensing a developing issue.

### Population Management User Interface

A user interface 301 of the data monitoring system 259 provides functionality for managing a population of patients, as shown in FIG. 17. The data can be displayed in various formats, including aggregated figures, tables, grades 321, bar charts 323, and percentage changes 325, 327.

In one embodiment, the aggregate metrics 317 can be calculated and displayed for all users associated with a specific healthcare provider, showing metrics like the total number of patients, average compliance, and the rate of detected abnormalities over various timeframes. Metrics from individual users within specific groupings can be displayed as population tabular metrics (319), which can be sorted by any of the included metrics.

### Care Team Communication and Data Security

As shown in FIG. 1, a communication module 270 can be provided from the data monitoring system 259 to the care team 151 regarding potential abnormalities or the status of a patient population. This communication may contain an abnormality alert 272 and/or abnormality data 274 and can be generated by either a processor or a person. Transmission can be accomplished through conventional means such as telephone, email, SMS, push notification, or a status LED on the device.

To ensure interoperability, one-way or bi-directional communication with an Electronic Health Record (EHR) may be provided via appropriate Application Programming Interfaces (APIs) and web standards like Health Level 7 (HL7), Fast Healthcare Interoperability Resources (FHIR), or Consolidated Clinical Document Architecture (C-CDA). To reduce the risk of breaches of electronic Protected Health Information (ePHI), various security methods may be employed, including Multi-Factor Authentication (MFA), firewalls Encryption-at-Rest, and Encryption-in-Transit.

In one embodiment, this communication can be used to address a change in the loading pattern of a foot caused by the formation of a callus. Information regarding the size and position of the callus can be provided to an orthotic manufacturer, who can then create and send a custom orthotic to the patient. This provides pressure offloading at the location of the callus, allowing it to resolve without requiring a visit to a healthcare facility.

### Annotation Tools and Data Generation

As described, an annotation pane 350, shown in FIG. 18, may be provided to enable the annotation of features seen in scans. This annotated data can be used to generate training data for algorithms that inspect scans. The annotation pane can be a standalone interface or integrated into other UI sections, such as the scan inspection pane shown in FIG. 14.

The annotation pane includes a filter menu 351 that allows users to alter the appearance of the image by adjusting parameters like contrast, saturation, and hue, or by applying a greyscale filter. It may also include presets, such as a “Callus” filter to increase the visibility of calluses or a “Contact Regions” filter to highlight areas of blanching. A hide sensor toggle 352 may be provided to eliminate the visibility of the sensors in the image, which can be achieved by interpolating the surrounding, using a previously received scan to fill the area, or employing a machine learning algorithm to estimate the visual data beneath the sensor.

The annotation pane also provides the ability to tag or define certain features. A feature of interest can be highlighted by creating a boundary, for example by drawing a polygon around it. The map 220 can then be used to determine the position of this feature within the temperature sensor dataset. Tags may be applied to the highlighted feature to denote its anatomical position (e.g., hallux, heel), clinical nature (e.g., callus, ulcer, dry skin), or other characteristics (e.g., dirt, debris, scan artifact).

### Feature Detection and Metrics

Once a feature is highlighted, various metrics related to it can be measured, including its size, shape, area, color, uniformity, and temperature. The physical size of a feature can be determined using the rectilinear grid of sensors as fiducial markers or by leveraging knowledge of the device's geometry. In one embodiment, a whole-foot detection algorithm can be developed to isolate the foot from the background, which is advantageous for reducing image file size and eliminating irrelevant visual information.

All of this tagged feature information can be stored with the scan in the database and used as input data to train feature detection algorithms, such as those based on corner/edge detection, SIFT/SURF, Hough Transforms, or machine learning models like CNNs.

### Temporal and Point-in-Time Analysis

When deployed, these algorithms can operate in a point-in-time manner on individual scans or on a longitudinal basis, tracking a feature or metric over time as new scans are received. This allows for the monitoring of not just absolute values but also their rates of change. Abnormality alert thresholds can be configured for both absolute values and rates of change, and can even be variable. For example, an increase in the contact area of the foot might be expected if the patient's weight has also increased, but could indicate an abnormality if their weight is stable.

Longitudinal changes in metrics can be monitored using various methods, including Peak Detection, Trend Detection (e.g., linear regression), Anomaly Detection (e.g., isolation forest), Change Point Detection, and Pattern and Motif Detection (e.g., dynamic time warping).

### Human and Algorithmic Supervision

The level of human supervision required can be adjusted based on the accuracy and confidence in the algorithms. In one embodiment, a high level of human supervision is retained, with the algorithm only used to highlight areas of potential abnormalities for a person to review. In another embodiment, at higher levels of confidence, algorithms may independently complete inspections and highlight abnormalities for human confirmation and decision-making. As model accuracy increases further, the system can be configured to determine the appropriate intervention for a potential abnormality and communicate it directly to the care team.

### Temporal Feature Analysis

To facilitate the comparison of features across different days, it is beneficial to isolate and align a temporal sequence of images so that the feature appears in the same location throughout the sequence. As shown in FIG. 19(a), a callus 703 can be detected 701, isolated from the image 702, and aligned 704 across multiple frames. The time series 705 of the feature's metrics, such as its area, can then be analyzed. Another useful process, shown in FIG. 19(b), is image subtraction, where subtracting an image from Day 1 from an image from Day 2 can highlight changes that are difficult to perceive by eye.

### System Flexibility and Hardware Configuration

The skin inspection system is designed with significant flexibility in both its analysis processes and its data capture modes, allowing it to adapt to various operational contexts and user preferences.

### Flexible Analysis: Human or Algorithm

The system's inspection and analysis processes can be performed by a human operator, an automated algorithm, or a combination of both.
Human Inspection: A healthcare professional or other users can manually inspect the visual and thermal data, leveraging their expertise and intuition to identify abnormalities that might be missed by algorithms.
Algorithmic Inspection: Automated algorithms, such as machine learning models or computer vision techniques, can be trained to analyze the captured data, providing a consistent and objective method for detecting patterns, anomalies, or changes over time.
Combined Approach: The system can also operate in a hybrid mode, where an initial screening is performed by algorithms, and any cases flagged for potential issues are then reviewed by a human expert. This approach ensures both efficiency and accuracy, leveraging the strengths of each method.

### Flexible Data Capture: Combined or Individual Data Types

The system is also capable of capturing and analyzing both temperature (thermal) data and visual (image) data, either simultaneously or individually.
Temperature (Thermal) Data: This is captured using an array of temperature sensors embedded in, or affixed on, the transparent panel and helps identify temperature variations that may indicate inflammation or other abnormalities.
Visual (Image) Data: This is captured using image capture devices (cameras) and provides detailed information about the skin's surface, allowing for the identification of visible abnormalities like ulcers or calluses.

This flexibility allows the system to operate in various scenarios. For example, a healthcare professional could use both thermal and visual data for a comprehensive manual inspection (“Human+Temp+Visual”), or an automated system could analyze both data types and flag issues for review (“Algorithm+Temp+Visual”). In situations where visual inspection is not feasible (e.g., poor lighting), a professional could rely solely on thermal data (“Human+Temp Alone”). Conversely, an algorithm could be configured to analyze only visual images (“Algorithm+Visual Alone”). This adaptability ensures the system can provide comprehensive or specialized inspection capabilities based on the specific needs of the scenario.

### Hardware and Software Configuration

It will be appreciated that the device 100 includes one or more software modules programmed to implement these predefined functions. The device comprises various hardware and software components, including a user interface, a CPU in communication with a memory, and a communication interface. The CPU, which may be a single processor or a multi-processor core, functions to execute software instructions that are loaded and stored in the memory. The memory may be any suitable volatile or non-volatile computer-readable storage medium, such as RAM, a ha d drive, flash memory, or a rewritable optical disk, and may be fixed or removable.

### Exemplary Embodiment and Use Case

The present disclosure can be embodied in various forms. The following description details an exemplary embodiment and provides a use case to illustrate the method and system. This exemplary embodiment should not be construed as limiting the scope of the invention, but rather as a specific implementation as way of an example

In its broadest sense, the system comprises a first imaging sensor configured to capture an image dataset of an inspection area, and a second sensor array comprising a plurality of discrete sensor elements positioned within the field of view of the first imaging sensor. The system is controlled by a processor and a memory storing executable instructions. While the following use case describes a medical application, it is understood that the inspection area could be any surface, the target could be any object, and the sensors could be configured to measure a wide range of parameters.

### A System for Diabetic Foot Monitoring

To illustrate the method and system of the present disclosure, an exemplary embodiment is described below in the context of a use case: the longitudinal monitoring of a patient's foot for the early detection of diabetic foot ulcers (DFU).

The system comprises a skin inspection device 100 as shown in FIG. 2. The first imaging sensor is a camera 107 equipped with a wide-angle lens 121, which is configured to capture a warped image 208, as illustrated in FIG. 6. The second sensor array comprises a plurality of discrete temperature sensors 105, which in this embodiment are thermochromic liquid crystal (TLC) formations that change color in response to temperature variations. While this exemplary embodiment uses temperature sensors, it is understood that the second sensor array could be configured to measure other physiological parameters, such as pressure, impedance, or pH. The system is controlled by a processor 300 and a memory storing executable instructions.

Longitudinal Monitoring of a Patient's Foot

### Step 1: Baseline Scan and Reference Data Map Generation

On Day 1, a user performs a baseline scan to establish a reference point for future comparisons.
Capture: The user places their foot 101 on the device's inspection area on the transparent panel 102. The camera 107 captures a reference image dataset. This image dataset is a warped visual representation of the user's foot and the array of temperature sensors 105.
Detect and Index: The processor 300 processes the captured image. It automatically detects the current pixel coordinates 206 for each visible discrete sensor element 105. In this embodiment, the detection is performed using a machine learning model trained to recognize the visual features of the sensor elements. The processor then determines the current index 621 for each detected sensor element, corresponding to its known position in the sensor array.
Generate Map and Associate Data: The processor 300 generates a reference data map 220 by associating each sensor's determined index 621 with its detected pixel coordinates 623. Concurrently, the system receives a discrete dataset of temperature values 252 from the temperature sensors 105. The processor 300 uses the reference data map to associate these temperature values with their corresponding pixel coordinates on the image. This reference map and its associated data are stored. The data can be visualized on a graphical user interface (GUI) 301, for example, by generating a visual heatmap and overlaying it on the image dataset in the visual inspection pane 312.

### Step 2: Follow-Up Scan and Current Data Map Generation

On a subsequent day, the user performs another scan. Due to natural variation, the user's foot is placed in a slightly different position and orientation. The system captures a new current image dataset and generates a current data map using the same process as on Day 1.

### Step 3: Alignment of Current and Reference Data Maps

Access and Register: The processor 300 accesses the stored reference data map. It then performs an image registration process to align the current data map with the reference data map. In this embodiment, this is achieved by performing a key-point matching algorithm, as illustrated in FIG. 10. The algorithm uses the stable visual pattern of the sensor array 105 in both the current image 608 and reference image 601 to find corresponding key-points 603.
Generate and Apply Transform: From these matched key-points, the processor creates a transform 604, such as a homography matrix. This transform mathematically describes the adjustment required to correct for the variation in the foot's position. The processor then applies this transform 605 to the current data map, aligning its coordinate system with that of the reference data map. Alternatively, the detection of the sensor elements could be performed using a contour detection algorithm 614 to identify their shapes and locations as shown in FIG. 11.

### Step 4: Comparison, Identification, and Action

With the maps now aligned, an accurate temporal comparison is performed.
Compare Data: The processor 300 compares the aligned datasets. For temperature data, it calculates the temperature difference at each corresponding sensor location, which can be displayed in the temperature asymmetry table 305. For visual data, the processor can perform an image subtraction between the aligned images, as shown in FIG. 19(b), to highlight changes.
Identify Abnormality and Alert: If an identified temporal change, such as a localized temperature increase shown in the risk chart 335, exceeds a predefined threshold 333, the system generates an alert 272.
System Response: This entire process is performed by the system for identifying temporal changes, which comprises the image capture device 107, the array of temperature sensors 105, and the processor 300. Upon generating an alert 272, the system's communication module 270 transmits the alert to a remote device, such as a workstation used by the care team 151, enabling timely and informed clinical intervention.

### Step 5: Clinical Action and Bi-Directional Data Correlation

The system's communication module 270 transmits an alert 272 to a clinician's workstation. This is where the bi-directional utility of the data map becomes critical for efficient clinical review.

Visual-to-Sensor Lookup: Upon receiving the alert, the clinician opens the patient's scan in the graphical user interface (GUI) 301. While examining the visual inspection pane 312, the clinician may notice a small area of discoloration on the foot image. To investigate further, the clinician selects the pixel coordinate corresponding to this visual feature using the pointer 302. In response to this selection of a pixel coordinate, the system uses the aligned data map 220 to identify an index of a discrete sensor element 105 located at or near the selected pixel coordinate, as illustrated by the process in FIG. 16. This enables the immediate retrieval of a data value (the temperature 307) from the second sensor array corresponding to that specific visual location, which is then displayed in the temperature asymmetry table 305.

Sensor-to-Visual Lookup: Conversely, the system itself may have generated the alert based on identifying a specific sensor index with a temperature value exceeding a threshold in the temperature asymmetry table 305. In response to this identification of an index, the clinician can select that entry in the table. The system then uses the same data map 220 to identify the pixel coordinate in the image dataset corresponding to the identified index. This enables the visual inspection of the specific region on the foot by automatically highlighting the corresponding sensor 306 on the visual inspection pane 312, allowing the clinician to assess the skin's appearance at the exact location of the thermal anomaly.

This bi-directional correlation allows for a rapid and intuitive investigation of potential issues. The clinician can seamlessly switch between analyzing a visual feature to see its thermal signature and investigating a thermal anomaly to see its visual manifestation, leading to a more accurate and confident clinical assessment and enabling timely intervention.

### Longitudinal Tracking Across Multiple Cameras

A further exemplary embodiment illustrates the system's robustness in tracking a feature of interest even when it moves between the fields of view of different cameras over time.

### System Configuration and Calibration

The skin inspection device 100 for this exemplary embodiment is equipped with at least a first image capture device (e.g., a heel camera) and a second image capture device (e.g., a forefoot camera), as suggested in FIG. 20. During the initial device calibration, the system is calibrated not only for each camera individually but also to understand the precise spatial relationship between the cameras. It generates a unified coordinate system for the entire inspection area, allowing it to stitch the images from all cameras together into a single, cohesive image dataset.

### Feature Migration Between Scans

Day 1 (Reference Scan): A patient performs a scan. A feature of interest, such as a small callus, is identified on the patient's heel. In this scan, the feature is located entirely within the field of view of the first image capture device (the heel camera). The system generates a stitched reference image and a corresponding reference data map, noting the feature's location within the unified coordinate system.
Day 2 (Current Scan): The patient performs a second scan but places their foot slightly further forward on the skin device 100. The same callus is now located entirely within the field of view of the second image capture device (the forefoot camera). The system generates a new stitched current image and a current data map.

### Alignment and Comparison in the Unified Coordinate System:

The processor 300 performs the image registration process as previously described. Because both the reference and current data maps are based on the same unified coordinate system, the alignment process works seamlessly. It aligns the stitched current image with the stitched reference image, correctly identifying that the feature seen by the forefoot camera on Day 2 is the exact same anatomical feature seen by the heel camera on Day 1.

The system can then accurately compare the characteristics of the feature over time, such as a change in its size or temperature, and identify a temporal change. This capability ensures that longitudinal tracking is not defeated by variations in foot placement, even when those variations cause a feature to move between the fields of view of different physical cameras.

### Device Calibration and Map Generation

An exemplary embodiment is described below in the context of a use case: the one-time calibration of a skin inspection device 100 during its manufacturing and setup. This process accounts for minor physical variations between individual skin inspection devices 100.

The system comprises a skin inspection device 100, which includes a first imaging sensor (a camera 107) and a second sensor array (an array of temperature sensors 105). The system also includes a processor 300 and a memory storing executable instructions. This calibration process addresses the problem illustrated in FIG. 8, where manufacturing variations can cause the sensor array to appear in different locations within the image frame from one skin inspection device 100 to another.

### One-Time Device Calibration During Manufacturing

This exemplary use case describes the method for creating a static, permanent device calibration map for each unique skin inspection device 100 before it is deployed for use.

### Step 1: The Calibration Environment.

This method is performed once as a part of a manufacturing or setup process. The skin inspection device 100 is placed in a controlled environment, such as a calibration station on an assembly line. This environment provides consistent, known lighting and a fixed mounting position to ensure an accurate baseline measurement. The purpose of this one-time process is to create a unique map for each device, thereby accounting for manufacturing variations between different skin inspection devices 100.

### Step 2: Capturing the Calibration Image Dataset

With the device 100 in the controlled environment, the processor 300 initiates a calibration sequence. The first imaging sensor, camera 107, captures a calibration image dataset of the second sensor array 105. In this step, the target is not a user's foot, but the sensor array itself, viewed through the transparent panel 102. This establishes the precise location of every sensor element relative to the camera for that specific device.

### Step 3: Processing and Device Calibration Map Generation

The processor 300 then processes the calibration image dataset to automatically detect the pixel coordinates 206 corresponding to the locations of the visual representations of the plurality of discrete sensor elements 105.
For each detected sensor element 105, the processor determines an index 621, which corresponds to its known physical position in the sensor array.
Finally, the processor generates the device calibration map 220 by associating, for each sensor element, its determined index 621 with its detected pixel coordinates 623.

### Step 4: Storage and Subsequent Use

The generated map is now a static correlation that is permanently linked to this specific device, for example, by storing it in the device's non-volatile memory or linking it to the device's serial number in a cloud database 260.
This device calibration map is subsequently used by the skin inspection device to correlate data in a plurality of later-captured image datasets taken by end-users. It serves as the foundational “key” that allows the system to know, for every future scan, exactly where each
ture reading 252 should be placed on the corresponding visual image 254, regardless of where the user places their foot.
   Device with a Knife Edge Aperture

To illustrate the structure and function of the apparatus, an exemplary embodiment is described herein, with primary reference to FIG. 26. This embodiment details a skin inspection device specifically engineered to minimize image artifacts caused by internal light reflections, thereby improving the quality and reliability of the inspection. The skin inspection device comprises a housing 106 which supports a transparent panel 102. The transparent panel 102 defines an inspection area where a target, such as a region of a user's body, is placed for inspection. Positioned within the housing 106 is an image capture device 107 and an illumination source 122. The image capture device is configured to capture an image of the inspection area through the transparent panel 102. In this embodiment, the image capture device 107 comprises a wide-angle lens 121, which is configured to capture a large field of view when the device is in close proximity to the target. A cover 135 is positioned over the illumination source 122 to control the path of the light.

The core inventive feature of this embodiment lies in the geometry of the aperture within the cover 135. The aperture is characterized by a knife edge 134. As shown in FIG. 26(b), this knife edge is not a simple opening with vertical walls 133) like the one shown in the comparative FIG. 26(a). Instead, the knife edge 134 is configured to minimize the area of an indirect reflection on the transparent panel 102 specifically by reducing the height of any vertical surfaces exposed to the illumination source 122.

Structurally, the knife edge 134 is formed by walls of the cover 135 that converge and taper to a sharp edge directed towards the illumination source 122. In this embodiment, the aperture is defined between a pair of opposing knife edges within the cover. To ensure optimal performance and symmetrical illumination, a central axis of the illumination source 122 is substantially aligned with a central axis of the aperture. This entire geometric arrangement ensures that stray light rays from the illumination source are effectively controlled and directed away from any upper surface of the knife edge itself.

The technical result of this configuration is a significant reduction in image artifacts. As visually demonstrated by comparing FIG. 26(a) and FIG. 26(b), the minimized area of the indirect reflection L2 produced by the knife edge aperture is substantially less than the area of the indirect reflection L1 produced by the conventional aperture with vertical walls. This reduction in artifacts leads to a tangible benefit: an increased usable inspection area in the captured image.

To further enhance the performance of the device, this embodiment can incorporate several additional features. The transparent panel 102 may have an array of temperature sensors 105 provided thereon, which may be thermochromic liquid crystal (TLC) formations. To control stray ambient light, the cover can be comprised of a substantially opaque and light-absorbing material and may feature a textured surface finish 138 configured to diffuse or absorb light reflections, a feature also illustrated in the context of FIG. 27. The interior surfaces of the housing 106 may also have a low-reflectivity coating, and the housing may further comprise one or more additional baffles 132, 139 to absorb or redirect stray light. In the embodiment shown, the aperture is substantially circular to provide even illumination.

The device is controlled by a processor 300 operably coupled to the image capture device and illumination source, which is configured to analyze the captured image to detect skin abnormalities. The device may be part of a larger system that includes a data monitoring system 259 in communication therewith.

The corresponding method for reducing image artifacts involves illuminating the inspection area with the illumination source and passing the light through the knife edge aperture 134 to minimize the area of indirect reflection. Capturing an image with this method results in a captured image with fewer artifacts compared to an image captured using an aperture with vertical walls.

### Adaptive Imaging and Monitoring

To illustrate the method and system of the present disclosure, an exemplary embodiment is described below in the context of a use case: the adaptive imaging and monitoring of a patient's foot for the early detection of diabetic foot ulcers (DFU).

The system comprises a skin inspection device 100 and a remote data monitoring system 259 in communication with each other. The skin inspection device 100 includes an image capture device 107, an illumination source 122, and a processor 115. In this embodiment, the device also includes an array of temperature sensors 105. The remote data monitoring system 259 includes a processor 300, a database 260, and a communication module 270.

### Adaptive Imaging Via a Feedback Loop

This use case illustrates the method for operating the skin inspection device, which involves an intelligent feedback loop between the remote system and the local device to optimize image capture for a specific feature of interest.

### Step 1: Initial Scan and Feature Identification.

Pre-Scan Check: A patient 150 places their foot on the device 100. Before a full scan, the processor 115 performs a pre-scan check to identify any suboptimal conditions, such as incorrect foot placement or the presence of foreign objects. If an issue is detected, the device provides feedback to the user to enable correction.
User Identification and First Image Capture: The processor 115 identifies the user based on their foot shape and weight and links the scan data to their patient profile in the data monitoring system 259. The device then proceeds to capture a first image of the inspection area. This first image may be a standard visual inspection image 254.

### Step 2: Remote Analysis and Risk Assessment

Data Transmission: The captured first image and associated temperature data 252 are transmitted to the remote data monitoring system 259.
Feature Analysis: The remote processor 300 analyzes the first image to identify a feature of interest. This feature could be a discrete object like a callus or, alternatively, a statistical property of a region, such as an average color value indicating redness. The processor considers one or more characteristics of the feature, such as its location, size, shape, and color.
Risk Assessment: The processor 300 then determines an assessed risk level associated with the feature. In this embodiment, the risk level is derived from a combination of the visual image data and the temperature data. For example, a discolored area that also shows a high temperature reading would be assigned a high risk level. This risk assessment can also be performed using a method of dynamically adjusting an alert threshold for one metric (e.g., temperature) based on a change in another metric (e.g., weight). In further embodiments only visual or only temperature data could be used to derive the risk level, combined optionally with other device or patient data.

### Step 3: The Feedback Loop-Adjusting Image Acquisition Parameters

Parameter Determination: Based on the high risk level, the remote processor 300 determines a set of adjusted image acquisition parameters. The goal is to get a better, more detailed look at the risky feature. These parameters are chosen based on the intended purpose of the second image, which is feature inspection.
Command Transmission: The remote system 259 then sends a command back to the skin inspection device 100 via the feedback loop. This command instructs the device's local processor 115 to use the new, adjusted parameters for the next image capture.

### Step 4: Capturing the Optimized Second Image

Parameter Adjustment: The local processor 115 receives the command and adjusts the device's settings. The adjusted image acquisition parameters may include increasing the illumination intensity or changing the exposure time, ISO, contrast, or color temperature. Specifically, the processor may program the illumination driver 128 to increase the illumination intensity only in the region corresponding to the location of the feature of interest.
Capture Second Image: The device 100 then captures a second image, which is a feature inspection image 255, using these new parameters. The adjusted parameters are specifically configured to optimize the visibility of the identified feature of interest in this second image.

### Step 5: Clinical Review and Reporting

Clinical Review: This entire workflow enables a more effective clinical review process. The clinician at the remote system receives an alert 272 and is presented with both the first image and the new, optimized second image.
Data Report: The processor 300 can analyze this second, higher-quality image to extract more precise metrics and generate a data report that includes these metrics along with the specific acquisition parameters that were used to capture it.
GUI Tools: The clinician can use a GUI 301 with an annotation pane 350 that includes a filter menu 351 to further enhance the image, apply presets like a “Callus” filter, and annotate the feature for the patient's record.

This adaptive feedback loop ensures that when a potential issue is detected, the system intelligently responds by capturing higher-quality, targeted data, leading to more accurate diagnoses and timely interventions. The entire process can be stored on a non-transitory computer-readable medium as executable instructions.

### Adaptive Imaging in a Multi-Camera System

To further illustrate the advanced capabilities of the present disclosure, this exemplary embodiment describes the operation of the adaptive imaging method in a skin inspection device equipped with multiple cameras. This use case demonstrates the system's ability to perform targeted, regional optimization by identifying which specific camera is viewing a feature of interest and adjusting only that camera's acquisition parameters.

### System Configuration

The system comprises a skin inspection device 100, as suggested in FIG. 20, which is equipped with at least a first image capture device 107 (e.g., a “heel camera”) and a second image capture device 107 (e.g., a “forefoot camera”). The system is controlled by a local processor 115 and a remote processor 300, which work together via a communication feedback loop. The system is calibrated to understand the spatial relationship between the cameras, allowing it to combine their image data into a single, unified “stitched” image.

### Targeted Feature Re-Acquisition

### Step 1: Initial Multi-Camera Scan

A patient 150 places their foot on the skin inspection device 100. The system performs an initial scan, capturing image data from both the heel camera and the forefoot camera.
The processor 300 combines this data to generate a stitched first image, which provides a complete visual representation of the entire plantar surface of the foot.

### Step 2: Analysis and Feature Localization

The processor 300 analyzes this stitched first image to identify a feature of interest. In this scenario, it detects a small, low-contrast area of discoloration on the patient's heel.
Crucially, by analyzing the feature's position within the unified coordinate system of the stitched image, the processor determines that the feature of interest is located within the field of view of the first image capture device (the heel camera).

### Step 3: The Adaptive Feedback Loop for Targeted Optimization

The system assesses the feature and determines that a higher-quality image is needed for accurate diagnosis. It determines a set of adjusted image acquisition parameters specifically designed to enhance the visibility of a low-contrast lesion (e.g., by increasing exposure time and adjusting the illumination 122.

The remote system 259 sends a command back to the skin inspection device 100. This command does not instruct a general re-scan; it contains targeted instructions.

### Step 4: Selective Capture of the Second, Optimized Image.

The device's local processor 115 receives the command. Based on the instructions, it controls only the first image capture device (the heel camera) to capture the second image using the new, adjusted image acquisition parameters. The second image capture device (the forefoot camera) is not instructed to re-capture an image, as the feature of interest is not in its field of view.
This results in a new feature inspection image 255 that is a high-quality, targeted, and optimized view of just the heel lesion.

By performing this selective re-acquisition, the system intelligently focuses its resources to get the best possible diagnostic data for the specific area of concern, without unnecessarily recapturing or altering the imaging of healthy areas. This targeted, adaptive approach in a multi-camera environment represents a significant improvement in efficiency and diagnostic precision.

### User Baseline Registration Process

To illustrate a further aspect of the method and system of the present disclosure, this exemplary embodiment describes a user baseline registration process. This process is typically performed during the initial setup of the skin inspection device for a new user and is designed to create a personalized anatomical and clinical baseline. This baseline model enables the system to perform more accurate longitudinal monitoring by distinguishing between genuine clinical changes and apparent changes that are merely due to variations in foot placement.

The system comprises the skin inspection device 100 and the data monitoring system 259, controlled by a processor 300.

### Initial User Registration and Baseline Model Generation

### Step 1: Initiation of the Registration Process

When a new patient 150 first uses the skin inspection device 100, the system recognizes that no baseline exists for this user. It initiates a guided baseline registration workflow, which is presented to the user on a graphical user interface (GUI) 301, which may be on an integrated screen or a connected mobile device.

### Step 2: Guided Multi-Position Scan Capture

The system instructs the user to perform multiple scans with their feet placed in various different positions and orientations on the inspection area of the transparent panel 102.
The GUI 301 may provide visual prompts, such as, “Please place your heel in the top-left corner,” followed by, “Now, please shift your weight to the outside of your foot.”
For each guided position, the device 100 captures a complete scan 250, including visual data 254 and temperature data 252. This captures the user's anatomical features as viewed from different locations and angles relative to the image capture device 107. This is crucial because, as illustrated in FIG. 8, the position of the target within the field of view 213 can significantly alter its appearance due to optical effects like compression and variations in illumination.

### Step 3: Data Processing and Baseline Model Generation

The processor 300 collects and analyzes this series of registration scans. It builds a personalized baseline model for the patient 150. This model serves two functions:
   1. Characterizing Positional Variation: The model learns how the appearance (size, shape, color) of the patient's specific anatomical features changes as a function of their position within the captured image. For example, it learns how a pre-existing callus appears to stretch or compress when the foot is rotated. This creates a “fingerprint” of the user's foot under different viewing conditions.
   2. Establishing a Clinical Baseline: The model records the patient's initial clinical state. A clinician, using the annotation pane 350 shown in FIG. 18, can review the registration scans and tag pre-existing conditions. For instance, a benign surgical scar that might otherwise be flagged as a new callus can be annotated as “pre-existing scar tissue.” Similarly, the system can analyze the temperature data 252 from all registration scans to establish a baseline temperature asymmetry profile, accounting for any chronic differences in blood perfusion between the feet due to conditions like peripheral arterial disease.

### Step 4: Subsequent Use of the Baseline Model in Longitudinal Monitoring.

Once the baseline registration is complete, the personalized model is stored and used in all subsequent daily scans.
When the system performs its temporal comparison (as described in the previous use case and illustrated in FIG. 19, it can now compare a new scan not just to the previous day's scan, but to the comprehensive baseline model.
If a change in a feature's appearance is detected, the processor 300 can consult the model to disambiguate the cause. It can determine if the change is consistent with the learned variations caused by a shift in foot position, or if it is a novel change that cannot be explained by positioning alone, thus indicating a potential clinical deterioration.

By performing this initial baseline registration process, the system becomes personalized to each user, significantly improving the accuracy and reliability of its temporal analysis and its ability to detect clinically relevant changes.

### User Identification in a Multi-User Environment

To illustrate a further aspect of the system's intelligence and utility, this exemplary embodiment describes a method for automatically identifying a user in a multi-user environment and ensuring their scan data is correctly logged to their personal profile. This process leverages the personalized baseline model created during the initial registration (as described previously) to act as a unique “fingerprint” for each user.

### The Problem: Data Integrity in a Multi-User Household

Consider a household where multiple individuals may use the same skin inspection device 100. For example, one user, “John,” may be a patient with diabetes who requires daily monitoring, while his spouse, “Jane,” does not. If Jane uses the skin inspection device 100, it is crucial that her scan data is not accidentally mixed with John's, as this would corrupt John's longitudinal data and could lead to false alerts or missed diagnoses.

### Automated User Identification and Data Routing

### Step 1: The Initial Registration “Fingerprint”

As described in the previous embodiment, both John and Jane complete a one-time baseline registration process. The system captures multiple scans of their feet in various positions and orientations.

The processor 300 analyzes these scans and generates a unique baseline model, or “fingerprint,” for each user. This model contains a rich set of data characterizing each user's specific anatomical features, such as:
Foot size and shape.
The unique pattern and texture of their skin.
The location of permanent features like scars or moles.
Their baseline weight.
Their baseline temperature asymmetry profile.

### Step 2: Performing a Scan in a Multi-User Environment

On a subsequent day, an unidentified user steps on the device 100.

The device captures a complete scan 250, including the visual image 254, temperature data 252, and weight data 256.

### Step 3: User Identification via “Fingerprint” Matching

Before logging the data, the processor 300 performs a user identification step. It compares the characteristics of the newly captured scan to the stored baseline “fingerprints” of all registered users (John and Jane).

The processor analyzes the foot size, shape, and unique visual features in the image 254 and compares them to the stored anatomical models. It also compares the captured weight 256 to the baseline weights.

Based on a high degree of correlation, the system identifies the current user. For example, if the foot shape and weight match John's stored profile, the system confidently identifies the user as John.

### Step 4: Correct Data Linking and Action

Once the user is identified, the system correctly routes the data.

If the user is John, the system links the captured scan data 250 to John's patient profile in the data monitoring system 259. The data is then used for his longitudinal analysis, and any detected abnormalities will trigger alerts for his care team 151. If the user is Jane, the system recognizes she is not the primary patient. It can be configured to take a different action. For example, it might simply display her weight on the device screen like a standard scale and discard the rest of the data, or it might log the weight to a separate fitness app, ensuring her data is never mixed with John's clinical record.

By using the detailed baseline model as a unique user fingerprint, the system ensures data integrity, enables personalized monitoring, and allows the device to be safely and effectively used in a multi-user household without compromising clinical accuracy.

### Real-Time User Guidance and Positional Correction

To illustrate a further aspect of the system's adaptive capabilities, this exemplary embodiment describes a method for providing real-time positional guidance to a user. This process uses the feedback loop to analyze a preliminary image, detect user placement errors, and provide corrective instructions before the final diagnostic scan is captured.
Initiate Pre-Scan: A user 150 places their foot on the inspection device 100. The device initiates a rapid, low-resolution “pre-scan,” capturing a first image of the foot's general position.
Analyze for Positional Deviation: The processor 300 analyzes this pre-scan image. Instead of looking for a clinical feature, it analyzes the position and orientation of the foot relative to a predefined optimal zone on the transparent panel 102. It detects that the user's foot is positioned, for example, too far forward and is slightly rotated.
Determine Corrective Instruction: Based on this positional deviation, the processor determines a set of corrective instructions.
Feedback Loop for User Guidance: The processor transmits a command back to the skin inspection device 100. This command does not adjust image parameters; instead, it instructs the skin inspection device 300 to provide feedback to the user. This feedback could be a message on a GUI 301 (“Please move your foot back and to the right”) or by activating specific guidance LEDs on the device itself.
User Correction and Final Capture: The user 150 sees the feedback and corrects their foot placement. Once the processor confirms the foot is in the optimal position, it proceeds to capture the high-quality, final diagnostic image.

This exemplary embodiment improves the quality and consistency of every scan by actively preventing user error, rather than just reacting to it.

### Automated Environmental Calibration and Correction

This exemplary embodiment describes a method for automatically calibrating image acquisition parameters based on the specific environment in which the device is being used. This ensures high-quality image capture even in uncontrolled settings like a user's home.
Capture Environmental Image: Before the user steps on the skin inspection device, or during a pre-scan check, the image capture device 107 captures a first image of the inspection area.
Analyze for Environmental Conditions: The processor 300 analyzes this image not for a clinical feature, but for environmental conditions. It may detect that the ambient light in the room is very low, or it may identify a strong specular reflection on the transparent panel 102 caused by an overhead room light.
Determine Compensatory Parameters: Based on this environmental analysis, the processor determines that the standard image acquisition parameters would result in a poor-quality image (e.g., underexposed or with significant glare). It calculates a set of compensatory acquisition parameters.
Feedback Loop for Environmental Correction: The processor sends a command to the device 100 to use these new, compensatory parameters. For example, if the room is too dark, the command might instruct the device to increase the intensity of its own internal illumination source 122. If there is a strong glare source, the command might instruct the device to use a much shorter exposure time to minimize the artifact's impact.
Capture Corrected Image: The device 100 then proceeds with the user scan, capturing the final image using the dynamically adjusted, compensatory parameters, resulting in a high-quality image despite the suboptimal environment.

This exemplary embodiment makes the skin inspection device 100 more reliable and “smarter,” allowing it to produce consistent results across a wide range of real-world conditions.

### AI-Assisted DFU Prevention and Model Improvement

To illustrate the method and system for training a machine learning model, this exemplary embodiment is described in the context of early detection and prevention of Diabetic Foot Ulcers (DFU) through a human-in-the-loop feedback system.

The system comprises a skin inspection device 100, a remote data monitoring system 259 with a processor 300, and a graphical user interface (GUI) 301. The system is designed to perform the method for training a machine learning model and can be stored as instructions on a non-transitory computer-readable medium.

### Use Case: Improving an AI Model for Early DFU Detection

### Step 1: Initial Scan and Automated Analysis

A high-risk patient 150 with known neuropathy performs a routine daily scan using the skin inspection device 100. The device captures a complete scan 250, including a visual inspection image 254 and temperature data 252.
The system's current machine learning model (“Version 1.0”) analyzes the data. It detects a minor thermal anomaly but is unable to classify it with high confidence, flagging it simply as a “potential feature of interest” for human review.

### Step 2: Expert Clinical Review and Annotation

A clinician at the remote data monitoring system 259 receives the flagged scan on their GUI 301. They open the image in the annotation pane 350.
The clinician, leveraging their expertise, recognizes the subtle signs of a developing problem. They see not just a hotspot in the temperature data, but also a corresponding faint area of redness and slight swelling in the visual image-a classic pre-ulcerative state that often precedes a DFU.
Using the polygon tool from the annotation tools 353, the clinician draws a precise boundary around this high-risk area. They then apply a label 354, classifying the feature's clinical nature as “Pre-ulcerative Lesion”.
Step 3: Generation of an Enriched Training Data Record

### Step 3: Generation of an Enriched Training Data Record

The processor 300 generates a new training data record based on the clinician's input. This record is highly enriched and contains multiple layers of information:
The user-generated annotation: the boundary coordinates and the “Pre-ulcerative Lesion” label.
The set of image acquisition parameters used to capture the image, such as the exposure time and illumination intensity.
Positional Data: The processor also determines the position and orientation of the user's foot within the inspection area for that specific scan.

### Step 4: Model Retraining and Deployment

The machine learning model is retrained using this new, enriched training data record. By including the acquisition parameters and positional data, the model learns to associate the specific visual and thermal signature of a pre-ulcerative state under various viewing conditions. It learns what this high-risk condition looks like, even when distorted by the wide-angle lens at the edge of the image.
This retrained, more intelligent model (“Version 1.1”) is then deployed back into the system.

### Step 5: Improved, Proactive DFU Prevention

At a later date, a different patient performs a scan that exhibits similar subtle signs of inflammation.
The new, retrained model analyzes the image. Instead of a generic flag, it now has the capability to identify the specific pattern it learned. It analyzes the subsequent image dataset and automatically highlights the area with high confidence, generating a specific alert: “High-Risk Pre-ulcerative Lesion Detected.”
This specific, actionable alert is sent to the care team 151, allowing them to intervene immediately with offloading instructions or to schedule a priority clinical review. This proactive intervention, made possible by the human-in-the-loop training cycle, helps to prevent the lesion from ever developing into a full-blown diabetic foot ulcer, demonstrating a significant improvement in clinical outcomes.

### Human-in-the-Loop AI Training

To illustrate the method and system for training a machine learning model, an exemplary embodiment is described below in the context of a use case: the continuous improvement of an AI algorithm for detecting skin abnormalities through a human-in-the-loop feedback system.

The system comprises a skin inspection device 100, a remote data monitoring system 259 with a processor 300, and a graphical user interface (GUI) 301. The system is designed to perform the method for training a machine learning model and can be stored as instructions on a non-transitory computer-readable medium.

### Use Case: Improving an AI Model for Callus Detection

### Step 1: Initial Automated Analysis and Clinician Review

The system begins with a first version of a machine learning model trained to detect common skin features. An image dataset is captured by a skin inspection device 100 and sent to the remote data monitoring system 259.
The processor 300, running this first version of the model, analyzes the image and automatically highlights a potential feature of interest.
This image, along with the model's initial finding, is displayed on a graphical user interface (GUI) 301 for review by a human expert, such as a clinician (as in claim 20).

### Step 2: Human Annotation and Correction.

The clinician examines the image on the annotation pane 350. They notice that the model has incorrectly identified a benign scar as a callus. The clinician uses the GUI's tools to correct this.
First, the clinician may use the filter menu 351 to adjust the image's contrast or apply a predefined filter to get a clearer view.
Then, using an annotation tool like the polygon tool, the clinician receives a user-generated annotation by drawing a precise boundary around the actual callus, which the model had missed.
The clinician then applies a label to this new boundary, identifying the feature's clinical nature as a “callus” and its anatomical position as the “heel”.

### Step 3: Generation of an Enriched Training Data Record

The system doesn't just save the boundary and label. The processor 300 generates a training data record that includes the clinician's annotation (the boundary and label) and the specific set of image acquisition parameters used by the device 100 to capture that particular image.
This set of parameters could include the exposure time, ISO, illumination intensity, or colour temperature.

Crucially, the processor also analyzes the image to determine the position and orientation of the user's foot within the inspection area and includes this positional data as part of the training data record. This is important because the visual appearance of a feature can be distorted by the wide-angle lens depending on its location within the image, as illustrated in FIGS. 6 and 7.

### Step 4: Model Retraining and Deployment.

This new, enriched training data record is then provided to the machine learning model for retraining. The processor 300 retrains the model using this new training data record.
By including both the acquisition parameters and the positional data, the model learns to identify a “callus” not just by its intrinsic appearance, but also how its appearance changes when viewed from different angles or under different lighting conditions. This enables the retrained model to accurately identify the feature of interest in subsequent images captured under different sets of image acquisition parameters and with the foot in different positions.
Once retrained, this second, improved version of the machine learning model is deployed back into the system.

### Step 5: Improved Automated Analysis

When a new image dataset is received, the system now uses this retrained model for its analysis. The improved model can now more accurately highlight potential calluses for review by the clinician (as in claim 6), having learned to distinguish between a true clinical change and a simple change in appearance caused by a different foot placement. This creates a continuous improvement cycle where human expertise makes the system's automated analysis progressively smarter and more reliable.
   Episode (of feature/abnormality)
   Features and/or abnormalities will persist in time, across multiple scans. Sometimes abnormalities will improve and disappear and may reoccur in future. It is therefore useful to classify episodes of abnormalities. The episode could be specific to an abnormality such as an area of callus, or in combination with other relevant data such as compliance, other health issues, daily life activities such as a holiday period, moving home etc.

### Regions/Segmentation of the Foot

The area of the foot may be divided into different regions which is useful for classification of features detected with respect to their anatomical location. For example, as described by Teymouri et al; hallux, second toe, little toes, medial forefoot, central forefoot, lateral forefoot, medial midfoot, lateral midfoot, medial heel, and lateral heel. The risk profiling of features/abnormalities may be related to their regional location. For example, a callus of 1 cm squared on the lateral heel may have a different risk profile that of the medial midfoot.

### Additional Inputs to a Data Monitoring System 259

Additional inputs may be provided into a data monitoring system 259 from sources other than scans from a skin inspection device 100. Other data include from external sources such as Electronic Health Records or from manual sources such as notes made on the patient account profile. Of particular relevance are data which may alter the risk profile or risk threshold of a user. For example, knowledge of a patient being on a particular medication may increase or decrease their risk threshold. Knowledge of other medical conditions or events such as being at risk of cardiac failure may modify the thresholds applied to weight monitoring. Other inputs include data from other monitoring systems, such as HbA1c (glycated haemoglobin), or Continuous Glucose Monitoring (CGM) levels.

### Automated Foot Inspection System

FIG. 28 is a flowchart illustrating a method for remote monitoring using multimodal data processing. The method facilitates the transformation of acquired scan data into an actionable clinical output, which may be used for detecting early signs of tissue stress or injury and initiating a clinical response.

The process is initiated with the acquisition of a scan 250, for example, from a home-based skin inspection device 100. During the scan, a set of multimodal data inputs 5002 is captured. The set of data inputs 5002 may comprise visual image data 254, temperature data 252, weight data 256, and compliance data 5004. Additionally, contextual data such as patient history 5003 is accessed and may be associated with the captured data inputs.

The data inputs 5002 are then provided to a processing module 5008. The processing module 5008 is configured to process each data input stream to generate a corresponding set of feature outputs 5009. For instance, image data 254 is processed to generate image features, temperature data 252 is processed to generate temperature features, and patient history 5003 is processed to generate clinical features.

The generated feature outputs 5009 are subsequently directed to one or more multimodal processing modules 5007. These modules are configured to fuse or correlate data from the different feature sets. For example, the multimodal processing module 5006 may associate image features with corresponding temperature features based on location, thereby correlating a visual abnormality with a thermal anomaly. This fusion of data from disparate sources enables a more comprehensive assessment of the patient's condition.

The output of the multimodal processing modules is provided to a clinical risk and contextualization generation module 5011. This module 5011 is configured to analyze the fused data to generate at least two outputs: a clinical risk output 5005 and a clinical context output 5010. The clinical risk output 5005 may be a quantifiable metric, such as a numerical risk score. The clinical context output 5010 provides supporting information or a rationale for the determined clinical risk.

Finally, the clinical risk 5007 and clinical context 5010 are used to determine and initiate a clinical action or communication 270, thereby facilitating a timely and appropriate response to a detected health risk.

This exemplary embodiment describes a method for an automated foot inspection system leveraging a skin inspection system, a series of automated analysis functions, analysis of recent captured scan data as well as other data about the patient (historic data, behaviour data, medical history, age etc), using the results of the analysis to automatically determine appropriate escalation or follow up, triggering an automated workflow based on the results. The system analyses data captured from the skin inspection system, as well as other data about the patient, such as their history and their behaviour. Based on results of these analyses, and a classification of foot health risk, a follow up action or workflow is triggered. This follow up action could be, for example, an automated message or report sent to the patient or health care provider, an escalation for review by a human reviewer, or the triggering of a workflow to contact the patient.

This allows for high volumes of patients to be monitored effectively and efficiently and increases number of patients that can be manager per human reviewer.
1. Automated Analysis Functions
   a. Full Automated Analysis: To carry out automated analysis, the described system uses an AI/ML algorithm, or algorithms, that are capable of [doing everything below and more, describe in more detail]. In another embodiment the system will use a series of algorithms including machine learning algorithms, computer vision algorithms, classical algorithms and human input to automate, or largely automate, this analysis.
   b. Screening for Issues: While many scans will have some risk present many will have no risk, this feature screens images for risk. It can be difficult to develop and secure regulatory approval for an algorithm that can identify and classify specific foot issues. A screening algorithm such as this, which focuses on identifying healthy feet will have lower development and regulatory burden.
      i. AI-Based Feature Extraction
         1. The image is processed by an artificial intelligence model, such as a convolutional neural network, transformer-based model, or ensemble classifier, trained to recognize visual features of foot anatomy and external objects.
      ii. Risk Assessment
         1. The AI model determines whether the foot presents no risk or whether a risk is present. Risks may include but are not limited to: presence of ulcers, wounds, swelling, discoloration, calluses, or other clinically relevant abnormalities.
      iii. Object/Condition Detection
         1. The AI model further determines whether the foot is covered or obscured by common external elements, including:
         a. Shoe detection: recognizing if footwear is present.
         b. Sock detection: recognizing if clothing is present.
         c. Bandage detection: recognizing if medical dressing is applied to the foot.
         d. Foreign matter detection: recognizing dirt, debris, or other substances on the skin surface.
      iv. Classification Output
         1. The system generates a classification output indicating (a) the risk status of the foot (no risk vs. risk present), and (b) the presence or absence of one or more covering elements (shoe, sock, bandage, dirt). This output may be displayed to a user, stored in memory, or transmitted to a remote monitoring system.
   c. Detection and Annotation of Issues
      i. AI model searches image identifies features and/or abnormalities and annotation, location of issue etc.
      ii. A first AI model searches for possible issues, searches for things that are abnormal or not usually present on healthy feet. It records location of the issue. A second model then looks at that location and determines what is there (is it an ulcer etc). In one embodiment, prior to the previous step, an algorithm modifies the image to normalise lighting, orientation, removes lens distortion, or de-warps the image so that the second model has more standardised images from which to analyse what it is looking at.
   d. Segmentation of foot into specific anatomical regions
      Why this is important: It is useful to know the regions of the foot in order to track changes over time, as an input to risk classification, and to perform advanced analysis. The following are various different approaches which can be used to segment the foot.
         i. AI Model Anatomical Segmentation
         1. Neural Network Processing
         a. The image is processed by a trained AI model, such as a convolutional neural network (CNN), transformer-based vision model, or a hybrid encoder-decoder architecture. The AI model is trained end-to-end on labeled datasets comprising foot images paired with corresponding region maps.
         2. Region Map Generation
         a. The AI model outputs a region map in which each pixel of the image is classified into one of a plurality of predefined regions. These regions may correspond to anatomical or functional areas, such as heel, arch, forefoot, toes, and lateral/medial zones. The output may be represented as a segmentation mask, probability map, or vectorized overlay.
         3. Post-Processing
         a. The system may refine the generated region map using morphological operations, geometric constraints, or statistical shape models to ensure anatomical plausibility and consistency across patients.
         ii. Using Keypoints
         a. Keypoint detection-Applying a machine learning and/or computer vision algorithm to the image to identify a plurality of keypoints corresponding to anatomical reference locations on the foot. Such keypoints may include, without limitation, the heel center, toe tips, metatarsal heads, ankle landmarks, and medial/lateral borders. The algorithm may be trained on a dataset of labeled foot images using supervised, unsupervised, or deep learning techniques, and may incorporate convolutional neural networks, keypoint detection networks, or geometric feature extraction methods.
         2. Geometric Mapping
         a. Computing a geometric transformation of the foot image based on the detected keypoints. This may include normalizing the image orientation, scaling to a reference size, or fitting the keypoints to a predetermined anatomical template.
         3. Overlay Generation
         a. Optionally, constructing an overlay comprising a plurality of region boundaries that correspond to predefined zones of the foot. The overlay is registered to the image by aligning template reference points to the detected keypoints. The overlay may define, for example, forefoot, midfoot, hindfoot, toe regions, plantar arch zones, or other clinically relevant subdivisions.
   e. Analysis of the characteristics of issues
      i. AI is trained to determine the characteristics of identified issues such as the size, color, shape etc, including changes to these characteristics over time
      ii. A human reviewer initially identifies and characterises an issue. In subsequent scans an algorithm determines if this issue is still present and if it is it automatically applies the same annotation as the previous scan.
      iii. As above except the algorithm assess for changes in characteristics
   f. Episodic analysis and linking issues to previously identified issues
      i. AI determines if issue is linked to previous issue
      ii. Algorithm identifies issue, second algorithm identifies location, third algorithm charterises issue, algorithm reviews previous days for issues of a similar profile, if one is found the current issue and the previous issue are linked together as an episode
   g. Assessment of patient risk
      i. AI Automated
         1. Neural Network Processing
         a. The image is processed by a trained AI model, such as a convolutional neural network (CNN), transformer-based vision model, or a hybrid encoder-decoder architecture. The AI model is trained end-to-end on labeled datasets comprising foot images paired with corresponding patient risk scores.
         2. Severity Assessment
         a. The AI model outputs a patient risk score for the image and saves it into a database.
      ii. System Approach
         1. A series of algorithms, as outlined herein, to determine if abnormalities are present, the severity of these abnormalities, if they are part of an episode, if the abnormality is worsening, the location of the abnormality and other parameters as outlined in this document. This data is analysed by a subsequent algorithm which compares this data to pre-defined reference data to determine a patient risk score.
      iii. Based on this risk score, the system is capable of carrying out an action which may include automatically communicating with the patient or the healthcare provider, alerting another user of the system, entering data into a database, creating a workflow task for a human to carry out such as to call the patient, or not taking any action.
2. The Means by which these algorithms can be used on input data to generate these clinical relevant context (e.g. episodes or care) and risk are clearly illustrated in FIG. 28 and are further described below.
3. Input Data (5001)
   a. Scans from skin inspection device (256)
   b. Historical data from previous scans (5002)
   c. Patient medical history including engagement notes (5003)
   d. Patient behaviour (compliance) (5004)
   e. Historical risks indicators (5005)
   f. Patient Weight Data (256)
   g. Scan inspection metadata (258)
   h. Image data features extracted from scan image data comprising: (5006)
      i. pixel regions that have been classified by their anatomical regions e.g. heel, toes, left, right foot, medial lateral regions, hallux, second toe, small toes, metatarsal, confidence/probabilities of accurate classification.
      ii. Pixel regions that have been classified as, for example: the foot, foreground or background objects, callus, bandaging, clothing, dry skin, elongated toe-nails, areas of contact/non-contact with the surface of the skin inspection device, soiling, trauma, tissue damage, scar tissue, prior areas of interest, pre-existing image features, new areas of interest, confidence/probabilities of accurate classification of these features.
      iii. The visual characteristics of features extracted from scan image data, including dimensions, color, area, shape, texture, saturation, intensity.
4. Data Processing
   a. Input Data processing (5008) may be applied to any input data source to extract features as an output (5009) that may be used in combination with any other raw or processed input data as part of a downstream multimodal processing step (5007).
   b. Processing may be achieved
      i. Manually by selecting an area of interest and applying a classification label
      ii. using classical computer vision approaches such as edge detection, template matching or
      iii. using trained machine learning models based on segmentation models such as UNET to classify pixels e.g. for contact region classification.
   c. Types of processing include:
      i. Processing of point in time raw image data into image features (as outlined in input data
      ii. Processing of point in time data into statistical features such as max, min, variance, skewness, etc. e.g. temp sensor data from an array is presented as a range of value to understand the largest temperature gradient across the surface of the foot.
      iii. Processing of time series data into statistical features
         1. Mean/Median/Mode—average or central tendency.
         2. Variance/Standard Deviation—spread of temperature.
         3. Min/Max—extremes.
         4. Range=max−min.
         5. Skewness—asymmetry in distribution.
         6. Kurtosis
      iv. Processing of time series data into temporal/shape features
         1. Trend—overall upward or downward direction (slope of regression line).
         2. Seasonality/Periodicity—repeating daily, weekly, or yearly cycles.
         3. Autocorrelation (lag features)—how temperature at time t relates to past values.
         4. Rate of change—e.g., ΔT/Δt between consecutive points.
         5. Peak frequency—how often local maxima occur.
         6. Duration of hotspot/cold spots—number of consecutive hours above/below thresholds.
      v. Processing of time series data into frequency domain features
         1. Dominant frequency-strongest repeating cycle e.g. temperature gradient between well perfused and less perfused regions of the foot.
         2. Spectral energy-how variance is distributed across frequencies.
         3. Entropy of spectrum-regularity vs randomness.
      vi. Processing of time series data into clinically relevant features
         1. Daily min-max difference (diurnal cycle).
         2. Weekly averages
         3. Number of threshold crossings per week
5. Muli-Modal Data Processing (5007)
   a. Data may be processed alone or in combination with other input data, time series data or features from data processing in a multimodal processing step to further enhance the detection of clinical risks and provide clinical context that drives clinical decision making.
   b. By way of example: input data from one data source e.g. temperature data (252) may be combined with an extracted region feature to determine the temperature distribution within the hallux region of the foot. This may be further processed in a multi-modal processing step (5007), using time series analysis to determine how this regional temperature distribution varies between the left and right foot over time in order to derive a clinical risk index (5005) i.e., a recent increase in the temperature distribution in this area may indicate a spike in inflammation versus the same point on the contralateral foot. Furthermore, by combining clinical patient history (5003) and/or prior callusing features detected, the source of the inflammation may be automatically contextualized (5010) for a clinical communication (270) as possible callus due to a history of callus and localized inflammation in the region.
6. Clinical Context (5010) and Risk Generation
   a. This stage transforms raw inputs+processed features into interpretable events, risk indices, and decision support outputs. It bridges technical outputs (like pixel-level features or time-series stats) with clinical meaning (like early ulcer risk, infection suspicion, or patient compliance issues).
   b. Event Detection—Identify deviations from normal baseline:
      i. Local inflammation→recent rise in temperature in a region compared to contralateral foot.
      ii. Tissue breakdown→new irregular shape, texture change, or color variation.
      iii. Contact anomalies→consistent lack of contact in regions (patient behavior or device use issues).
      iv. Behavioral events→missed scans, reduced compliance, delayed healing markers.
   c. Clinical Feature Contextualization-Combines multimodal features into clinically interpretable patterns:
      i. Inflammation risk—Regional temperature spike+historical callus+medical history of diabetic foot ulcer.
      ii. Mechanical stress risk—Repeated hotspot under metatarsal heads+patient weight trend upward.
      iii. Infection suspicion—Abnormal redness (image features)+temperature elevation+prior wound notes.
      iv. Poor compliance detection—Missed scans+unchanged high-risk features+patient notes showing non-adherence
   d. Risk Index Generation-Quantify clinical risk as indices or scores, e.g.:—
      i. Regional Inflammation Risk Index (RIRI)→based on thermal asymmetry, trends, and contralateral comparison.
      ii. Tissue Breakdown Risk Index (TBRI)→based on texture/shape changes in skin integrity.
      iii. Compliance Risk Score (CRS)→based on scan frequency, behaviour notes, and device metadata.
      iv. Composite Clinical Risk Score (CCRS)→fusion of multiple modalities for overall patient risk
   e. Clinical Reporting & Decision Support-Reports for clinicians could include:
      i. Summary page: Key detected risks (e.g., “Rising inflammation in left hallux, probable callus-related”).
      ii. Trend visualizations: Graphs of temperature, weight, compliance over time.
      iii. Image overlays: Highlighted areas of tissue risk, change maps vs baseline.
      iv. Contextual notes: Linking detected patterns with patient history and clinician notes.
      v. Alerts & Flags:
         1. “High risk of ulcer formation within next 2 weeks”
         2. “Non-compliance detected→intervention recommended”
   f. Clinical Value
      i. Early intervention→flag risks before ulceration/infection occurs.
      ii. Personalized monitoring→risks linked to patient-specific history.
      iii. Improved compliance→behavioural insights encourage patient engagement.
      iv. Decision support→reduces cognitive burden on clinicians by highlighting the “why” and “where” of risk.
      v. Workflow automation→removes the need for manual analysis by clinician and facilitates population analysis and management across large patient populations
   g. Example Scenario
      i. Input: Temperature spike of +2.5° C. in left hallux, compared to contralateral, prior callus in the area detected using image feature extraction algorithms.
      ii. Contextualization: Patient history shows recurrent callus in same region. Risk index: High RIRI=0.85 (on scale 0-1).
      iii. Report output: “High-risk inflammation detected in left hallux. History of callus in same region suggests probable recurrence. Recommend further inspection and offloading intervention.”
7. Outputs
   a. Initiation of a workflow based on the outcome of holistic risk assessment
      i. Patient escalation—call, SMS, app alert, etc.
      ii. HCP escalation—emergency or standard
      iii. Internal escalation
      iv. No escalation but data is stored in patient profile to be used in future analysis etc.
   b. Based also on patient preferences and past behaviour
      i. Such as if they only want to receive a phone call, at what time of day, did we have success in contacting them at a certain time of day before, do we call working age people in the evening versus day time for older, do we escalate to HCP directly as patient can't ever be contacted by us etc.

### a Risk Assessment Method Based on Comparing the Characteristics of a Detected Feature, to the Predicted Characteristics of a Known Feature/Abnormality Based on Longitudinal Observations.

FIG. 29 is a flowchart illustrating a method for risk assessment of a detected feature based on a comparison of its observed characteristics to its predicted characteristics derived from longitudinal data. This method allows the system to distinguish between stable, lower-risk abnormalities and changing, higher-risk abnormalities.

The method is initiated upon receiving a scan 250. The scan data is processed by a feature detection algorithm 6001, which is configured to identify one or more features of interest within the scan. In the exemplary embodiment shown, the algorithm detects a first feature (A) 6002 and a second feature (B) 6003.

The detected features are then processed by an episodic feature matching algorithm 6005. This algorithm compares the characteristics of each detected feature to a set of pre-existing feature episodes 6007 stored in a database 260. These episodes represent the historical data of features tracked across multiple previous scans.

If the episodic feature matching algorithm 6005 does not find a match for a detected feature, such as for feature (A) 6002, the feature is classified as a new feature 6006. The risk associated with this new feature is then assessed based on its own observed characteristics.

If the algorithm 6005 finds a match for a detected feature, such as for feature (B) 6003, a different analytical path is taken to assess if the feature has changed over time. The matched feature data triggers a feature appearance prediction algorithm 6008. This prediction algorithm 6008 receives multiple inputs to generate its prediction. A first input is the historical data for the specific pre-existing episode 6007 corresponding to the matched feature, which may include its trajectory or rate of change over time. A second input is context data 6009, which can include positional data from the current feature detection 6001 (e.g., location and orientation of the feature in the scan 250) and other contextual information from the database 260 (e.g., time elapsed since the last scan).

The feature appearance prediction algorithm 6008 processes these inputs to generate a set of predicted feature characteristics 6010, which represent the expected state of the feature at the time of the current scan.

A difference assessment module 6012 then performs a comparison between the predicted feature characteristics 6010 and the observed feature characteristics 6011 (which are derived from the feature detection algorithm 6001 for the current scan).

The result of this comparison is used for risk triage. If the difference assessment 6012 determines there is a “Small difference” between the predicted and observed characteristics, the feature is classified as having a “Lower risk,” indicating it is stable or changing as expected. Conversely, if a “Large difference” is detected, the feature is classified as having a “Higher risk,” indicating an unexpected or accelerated change that may require intervention.

A challenge which arises when performing automated inspections of foot scans is being able to distinguish between pre-existing features/abnormalities which are stable and lower risk, and those which are changing and therefore higher risk. Many patients have pre-existing features/abnormalities which are consistently detected by feature detection algorithms. Examples can include an area of scar tissue, or a mole, an area of stable callus, or stable epithelialized wound. The features/abnormalities will be detected in each new scan received by a feature detection algorithm, and if compared to a static threshold will be determined to be high risk and cause a false positive assessment of their being of an issue requiring intervention. This can create inefficiencies for the monitoring team and may even cause a patient to lose confidence in the monitoring system if they are repeatedly being warned about an issue which is pre-existing and currently low risk.

As a result, it is very beneficial to have a means of distinguishing between pre-existing features/abnormalities which are stable, and those which are changing. This is achieved by the method described in FIG. 29. A scan 250 is received by a data monitoring system 259. A feature detection algorithm 6001 runs on the received image to detect the location of the foot within the image, and any features/abnormalities on the foot. In the example flowchart in FIG. 29, two features are detected within the scan 250; feature (A) 6002, and feature (B) 6003. Pre-existing episodes 6008 of features/abnormalities may exist for this patient. These pre-existing episodes are created by tracking and associating features/abnormalities across multiple scans, including from baseline data, by generating a map which links the location of the feature/abnormality through different scans.

An episodic feature matching algorithm 6005 assesses the characteristics of the features detected by the feature detection algorithm 6001 and checks for a match with pre-existing episodes 6007. This algorithm accounts for variation in the location of the foot across the received scans, that will modify the appearance of a feature/abnormality due to changes in visual distortion or illumination.

If a match is not found by the episodic feature matching algorithm 6005, this feature is classified as a new feature, and the risk is assessed on the basis of the observed characteristics of the feature/abnormality, and monitoring and/or intervention workflows may be triggered accordingly.

If a match is found, it is important to assess if the feature has changed to properly assess the risk level. This can be achieved by using a feature appearance predication algorithm 6008 to generate predicted feature characteristics 6010. Inputs to the feature appearance prediction algorithm 6008 include the episodic data 6007 from the detected feature 6003. This can include the trajectory of the appearance of that feature/abnormality over a period of time i.e., the rate of change of the feature/abnormality of a certain episode. For example, the predicted characteristics of a region of callus could include size, shape, colour, location, area, texture, appearance etc.

Additional inputs to the feature appearance prediction algorithm 6008 include additional context data 6009, including information sourced from the feature detection algorithm 6001 such as the location of the feature in the scan received 250, including the position of the foot and its orientation. The algorithm accounts for variation in the location of the foot in the received scans, updating the predicated appearance that will occur due to changes in optical distortion or illumination levels for example. Additional contextual data inputs 6009 to the feature appearance prediction algorithm 6008 include data from the patient record in the database 260, which includes context like time since last scan. This is used to estimate the predicted appearance by combining the rate of the change of appearance with amount of time that has elapsed since the last observation.

The output of the feature appearance prediction algorithm 6008 is the predicted feature characteristics 6010, and the outputs of the feature detection algorithm 6001 are the observed feature characteristics 6011. A difference assessment 6012 can then be performed on these two outputs. If a small difference is detected between the predicted feature characteristics 6010 and the observed feature characteristics 6011, this indicates that there is a low change is the characteristics of the feature/abnormality and therefore the level of risk is lower. Conversely, if there is a significant difference between the predicted feature characteristics 6010 and the observed feature characteristics 6011, this indicates that there is a large change in the characteristics of the feature/abnormality and therefore the level of risk is higher.

This approach allows more accurate determination of the risk levels of features detected within scans and therefore improves the efficiency and efficacy of clinical monitoring and intervention.

The example given is a simple triage based on there being a difference or not between the observed feature characteristics 6011 and the predicted feature characteristics 6010.

More sophisticated assessments can also be performed based on the contextual information.

For example, an area of epithelialized wound has been healing progressively over the past 10 days, resulting in a decrease in size of the feature, and a lightening of the colour. No scan is received for 5 days. The predicted feature characteristics 6010 of this feature at this time would be an improvement in appearance compared to the previous appearance, based on the healing trajectory of the episode and contextual information from the databased such as the typical rate of healing of features of this nature. In this example, if the observed feature characteristics 6011 may be similar to those observed in a previous scan, but when compared to predicted feature characteristics 6010 it becomes clear that the rate of healing is not in line with the expected value, and therefore the risk classification is higher.

AI-Assisted Risk Assessment with a Longitudinally-Trained Model

To illustrate the method and system of the present disclosure, an exemplary embodiment is described below in the context of an exemplary use case: the complete lifecycle of creating and using a sophisticated, longitudinally-trained machine learning model for the early detection and risk assessment of Diabetic Foot Ulcers (DFU).

The system comprises a skin inspection device 100 that captures scans 250, and a central data monitoring system 259 with a processor 300. The system includes a display screen and an input device for a graphical user interface (GUI) 301. The processor 300 and memory store the instructions and models necessary to perform the methods described herein. The entire system can be considered a system for AI-assisted clinical diagnosis, with its instructions stored on a non-transitory computer-readable medium.

Part 1: The Training Phase—Creating a Longitudinally-Aware AI Model Data Collection and Alignment: The system accesses a plurality of historical image datasets for a patient, captured over multiple scans. These datasets contain a specific feature of interest-a suspicious-looking area of redness, for example. The processor aligns the plurality of historical image datasets to a common reference frame using an image registration process, such as key-point matching, to correct for day-to-day variations in the user's foot placement. This creates a time-series of images showing the feature at a consistent anatomical location.

Expert Annotation and Contextual Data Generation: A clinician, acting as the user, reviews this aligned series of images on the GUI 301. Using the annotation tools on the annotation pane 350, the clinician draws a precise boundary around the feature in each image and applies a label identifying its clinical type as a “pre-ulcerative lesion”. The clinician can use the filter menu 351 to adjust image properties to ensure the annotation is accurate.

Enriched Training Package Generation: The processor 300 then generates a comprehensive training data package. For each annotated image, it creates a training record comprising:
1. The clinician's user-generated annotation (boundary and label).
2. The contextual data, which may include:
   The set of image acquisition parameters used to capture that specific image. This is vital because some of these images may have been captured with adaptively adjusted parameters to get a better view, and the model must learn what the feature looks like under these varying conditions.
   The positional data indicating where the feature was located in the original, unaligned image. This allows the model to learn the effects of optical distortion.

Training the Model: The machine learning model is then trained using this complete training data package. The model doesn't just learn what a pre-ulcerative lesion looks like in a single image; it learns the entire trajectory of how it evolves over time and how its appearance changes under different lighting conditions and at different positions on the foot. This entire process represents a human-in-the-loop feedback cycle where expert knowledge is used to progressively improve the model.

### Part 2: The Application Phase-Using the Trained Model for Risk Assessment

New Scan and Analysis: A new patient performs a scan, and the system captures a current image dataset. This dataset includes both visual data and corresponding thermal data.
Risk Assessment by the Trained Model: The new image is analyzed by the skin abnormality detection model to identify a current instance of a feature of interest. The model compares the current feature to the vast number of historical trajectories it has learned.
Determining the Risk Level: The processor, executing the model, determines a risk level for the feature. This determination is made by comparing the current instance of the feature with a predicted state that the model generates based on the patient's own historical data.
Scenario A (New Feature): If the model analyzes the image and determines that the feature does not correspond to any known feature in the patient's history, it can be configured to automatically assign a high-risk level and flag it for immediate review.
Scenario B (Known Feature): If the feature is a known pre-ulcerative lesion, the model predicts its expected state. If the current state matches the predicted trajectory (e.g., it is healing as expected), the risk level is determined to be low. If the current state deviates significantly from the predicted trajectory (e.g., it has grown larger or hotter when it was expected to shrink), the risk level is determined to be high.
Actionable Alert: Based on a high-risk determination, the system generates a clinically actionable alert and transmits it to the care team.

By training a model with aligned, longitudinal, and context-rich data, the system can perform a level of nuanced risk assessment that is impossible with simpler models, leading to more accurate diagnoses and better patient outcomes. In essence, this phrase describes a machine learning model that has been trained not just on what features look like, but on how they evolve over time and how their appearance is affected by the specific conditions under which they are observed. This creates a far more accurate, reliable, and clinically sophisticated diagnostic tool.

It is to be understood that throughout this disclosure, certain terms related to data processing may be used interchangeably. The terms “model,” “trained model,” “machine learning model,” “AI model,” “algorithm,” “feature detection algorithm,” “predictive model,” and similar phrases all refer to one or more data processing techniques that can be executed by a processor 300. These techniques are configured to receive input data, perform one or more computational steps, and generate a useful output. The use of any specific term should not be construed as limiting the invention to that particular type of computational method, as any suitable data processing technique that performs the described function is contemplated herein.

### Definition of Terms

To provide a clear and consistent understanding of the disclosure, certain terms used throughout this specification and the appended claims are defined below. It is to be understood that these definitions are provided to assist in the understanding of the invention and are not intended to be limiting.

Model, Algorithm, Engine, Module: These terms, including phrases like “Feature Detection Algorithm” or “Predictive Model,” are used interchangeably to refer to one or more data processing techniques that can be executed by a processor 300. These techniques are configured to receive input data, perform one or more computational steps, and generate a useful output. They are not limited to any specific type of computational method, such as neural networks, statistical regression, or rule-based systems.

Image Registration Process/Alignment: These terms refer to any computational process used to bring two or more images into the same coordinate system. The goal is to establish spatial correspondence between the images, such that the pixels representing the same anatomical location on a target are aligned, thereby correcting for variations in the target's position, orientation, or scale between captures.

Longitudinal Episode/Historical Data: These terms refer to a collection of data points and observations associated with a single, specific feature of interest that has been identified and tracked across a plurality of scans captured over a period of time. An episode contains the “life story” or trajectory of a feature.

“Contextual Data” is used as a general term to describe any data that provides additional information or context for a primary dataset, such as an image. For the purpose of this disclosure, contextual data can be broadly understood to comprise several categories, which may include, but are not limited to, Acquisition Context Data and Clinical Context Data

Acquisition Context Data: This term refers to any data that describes the technical conditions under which a primary dataset was captured. Its primary purpose is to allow the system to account for non-clinical variations in a feature's appearance. This includes, but is not limited to:
Image Acquisition Parameters: The specific settings of the image capture device, such as exposure time, ISO, illumination intensity, and white balance.
Positional Data: The location and orientation of a target body part within the inspection area.
Environmental Data: Ambient temperature or lighting conditions in the room where the scan was taken.

Clinical Context Data: This term refers to any data, other than the primary image and sensor data, that provides clinical, behavioural, or lifestyle context for a specific user. Its primary purpose is to allow the system to perform a more nuanced and personalized risk assessment. This includes, but is not limited to:
Historical Clinical Metrics: A user's baseline temperature asymmetry, their typical compliance levels with the scanning schedule, or the trajectory of previously monitored abnormalities.
Case Note Data: Information that may be manually entered by a clinician or the user, such as current medications, recent changes in activity levels, or significant life events (e.g., a recent illness).
User Profile Information: Demographic data and relevant medical history, such as the duration of diabetes or the presence of a known condition.

Training Data Record: This refers to a structured data element used for training a machine learning model. A single record comprises at least a portion of an image dataset, a user-generated annotation corresponding to that image data, and associated contextual data.

Annotation: This refers to information added to an image dataset by a user, typically a human expert. An annotation comprises at least a boundary (e.g., a set of coordinates defining a polygon or a bounding box) that delineates a feature of interest, and a label that identifies a type, classification, or characteristic of the feature within the boundary.

Skin Inspection System: This term is used broadly to encompass the entire operational architecture. It may refer to a standalone skin inspection device 100 that performs all processing locally, or it may refer to a distributed architecture comprising one or more skin inspection devices acting as clients in communication with a remote data monitoring system 259 that performs some or all of the data analysis.

User/Patient/Individual/Subject: These terms may be used interchangeably to refer to the person whose body part is being inspected by the device, without limiting the invention to a formal clinical or doctor-patient relationship.

It will be appreciated by the person of skill in the art that various modifications may be made to the above described embodiments without departing from the scope of the present disclosure. In this way it will be understood that the teaching is to be limited only insofar as is deemed necessary in the light of the appended claims. In the exemplary arrangement; multiple image capture devices are illustrated, however, it will be appreciated that a single image capture device may be used.

Similarly the words comprises/comprising when used in the specification are used to specify the presence of stated formations, integers, steps or components but do not preclude the presence or addition of one or more additional formations, integers, steps, components or groups thereof.

What is claimed is:

1. A method for operating a skin inspection device, the method comprising:
capturing a first image of an inspection area;
analyzing, by a processor, the first image to identify a feature of interest;
determining, by the processor, a set of adjusted image acquisition parameters based on one or more characteristics of the identified feature of interest; and
capturing a second image, being a feature inspection image, of at least a portion of the inspection area using the adjusted image acquisition parameters, wherein the adjusted image acquisition parameters are configured to optimize the visibility of the identified feature of interest in the second image.

2. The method of claim 1, wherein the first image is a visual inspection image or an image from a pre-scan check.

3. The method of claim 1, wherein the one or more characteristics of the feature of interest comprise at least one of: a location of the feature, a size of the feature, a shape of the feature, a color of the feature, or an assessed risk level associated with the feature, wherein the assessed risk level is derived from a combination of visual image data and temperature data.

4. The method of claim 1, wherein the adjusted image acquisition parameters comprise at least one of: an illumination intensity, an exposure time, an ISO setting, a contrast setting, a white balance setting, or a colour temperature.

5. The method of claim 4, wherein determining the set of adjusted image acquisition parameters comprises increasing the illumination intensity in a region corresponding to the location of the identified feature of interest by programming individual control of LEDs or groups of LEDs via driver circuitry.

6. The method of claim 1, further comprising capturing multiple images with different acquisition conditions for different regions within the inspection area, and combining optimized portions of these multiple images to create a high dynamic range (HDR) image, thereby enhancing the visibility of features across varying illumination levels within the inspection area.

7. The method of claim 1, wherein the adjusted image acquisition parameters are configured based on an intended purpose of the second image, wherein the purpose is selected from the group consisting of: providing a life-like appearance for visual inspection, increasing visibility of specific features for feature inspection, and minimising colour signal noise for temperature measurement.

8. The method of claim 1, wherein the skin inspection device comprises at least a first image capture device and a second image capture device, and wherein:
capturing the first image comprises generating a stitched first image from image data captured by the first and second image capture devices;
analyzing the first image comprises analyzing the stitched first image to determine that the feature of interest is located within a field of view of only the first image capture device; and
capturing the second image comprises controlling only the first image capture device to capture the second image using the adjusted image acquisition parameters.

9. A skin inspection device, comprising:
an image capture device configured to capture images of an inspection area;
an illumination source configured to illuminate the inspection area; and
a processor operatively coupled to the image capture device and the illumination source, the processor configured to:
control the image capture device to capture a first image of the inspection area;
analyze the first image to identify a feature of interest;
determine a set of adjusted image acquisition parameters based on a location or appearance of the identified feature of interest; and
control the image capture device or the illumination source to capture a second image of the inspection area using the adjusted image acquisition parameters to enhance the visibility of the feature of interest.

10. The skin inspection device of claim 9, wherein the processor is configured to determine the adjusted image acquisition parameters based on a risk level associated with the feature of interest, the risk level being received via a feedback loop from a remote data monitoring system.

11. The skin inspection device of claim 9, wherein the processor is configured to perform a pre-scan check to identify the feature of interest prior to capturing the first image, wherein the pre-scan check identifies conditions selected from the group consisting of: incorrect foot placement, the presence of foreign objects, and soiling on a transparent panel.

12. The skin inspection device of claim 9, wherein the processor is further configured to identify a user of the device based on one or more characteristics of the user, and to link captured scan data to the identified user's patient profile in a data monitoring system.

13. The skin inspection device of claim 9, further comprising a graphical user interface (GUI) with an annotation pane that includes a filter menu providing tools for adjusting visual properties of a displayed image and applying predefined filter settings to enhance the visibility of specific features.

14. The skin inspection device of claim 9, further comprising an illumination driver, wherein the processor is further configured to detect an illumination fault and, in response, to take a remedial action.

15. The skin inspection device of claim 9, further comprising a cover positioned over the illumination source, the cover defining a knife-edge aperture configured to reduce indirect reflections on a transparent panel.

16. A skin inspection system, comprising:
an image capture device;
an illumination source;
a processor; and
a memory storing instructions which, when executed by the processor, cause the system to perform a method for operating a skin inspection device, the method comprising:
capturing a first image of an inspection area;
analyzing, by a processor, the first image to identify a feature of interest;
determining, by the processor, a set of adjusted image acquisition parameters based on one or more characteristics of the identified feature of interest; and
capturing a second image, being a feature inspection image, of at least a portion of the inspection area using the adjusted image acquisition parameters, wherein the adjusted image acquisition parameters are configured to optimize the visibility of the identified feature of interest in the second image.

17. The system of claim 16, further comprising a temperature sensor array, wherein the processor is configured to analyze both image data from the image capture device and temperature data from the temperature sensor array.

18. A method for assessing abnormality risk in a skin inspection system, the method comprising:
monitoring a first patient metric derived from scan data;
monitoring a second, different patient metric derived from the scan data;
dynamically adjusting an abnormality alert threshold for the first patient metric based on a change in the second patient metric; and
generating an abnormality alert if the first patient metric exceeds the dynamically adjusted threshold.

19. The method of claim 18, wherein the first patient metric is a foot contact area and the second patient metric is a patient weight.

20. The method of claim 1, wherein the feature of interest is a statistical property of at least a portion of the first image, the statistical property selected from the group consisting of an average color value, a texture variance, and a brightness distribution.

21. The method of claim 1, further comprising:
analyzing the second image to extract one or more metrics related to the feature of interest; and
generating a data report comprising the one or more extracted metrics and the adjusted image acquisition parameters used to capture the second image.

22. A method for operating a skin inspection device, the method comprising:
performing one or more pre-scan checks to identify a suboptimal scan condition; and
providing feedback to a user, based on the identified suboptimal scan condition, to enable correction prior to or during a scan.

23. A skin inspection system comprising:
a skin inspection device comprising an image capture device configured to capture image data and having adjustable image acquisition parameters; and
a remote data monitoring system communicatively coupled to the skin inspection device, the remote data monitoring system comprising a processor configured to:
receive the image data from the skin inspection device;
analyze the image data to determine a risk level; and
transmit a command to the skin inspection device to adjust at least one of the image acquisition parameters based on the determined risk level.

24. A non-transitory computer-readable medium storing instructions that, when executed by a processor of a skin inspection system, cause the system to perform a method for operating a skin inspection device, the method comprising:
capturing a first image of an inspection area;
analyzing, by a processor, the first image to identify a feature of interest;
determining, by the processor, a set of adjusted image acquisition parameters based on one or more characteristics of the identified feature of interest; and
capturing a second image, being a feature inspection image, of at least a portion of the inspection area using the adjusted image acquisition parameters, wherein the adjusted image acquisition parameters are configured to optimize the visibility of the identified feature of interest in the second image.

25. A method of clinically reviewing a skin condition, the method comprising:
receiving, at a remote data monitoring system, an alert indicating a potential abnormality identified in a first image captured by a skin inspection device;
sending a command from the remote data monitoring system to the skin inspection device, the command instructing the device to adjust one or more image acquisition parameters to optimize visibility of a feature of interest associated with the alert; and
receiving a second image captured by the skin inspection device using the adjusted image acquisition parameters for further clinical review.

26. A method for operating a skin inspection device to compensate for environmental conditions, the method comprising:
capturing a first image of an inspection area to assess one or more environmental conditions;
analyzing, by a processor, the first image to identify a suboptimal environmental condition;
determining, by the processor, a set of compensatory image acquisition parameters configured to mitigate an effect of the suboptimal environmental condition; and
capturing a second, diagnostic image of a target in the inspection area using the compensatory image acquisition parameters.

27. The method of claim 26, wherein the suboptimal environmental condition is selected from the group consisting of a low ambient light level and a specular reflection on a transparent panel of the device, and wherein the compensatory image acquisition parameters comprise at least one of an increased illumination intensity or a reduced exposure time.
